(19) United States
(12) Patent Application Publication
(10) Pub. No.: US 2022/0054256 A1
(43) Pub. Date: Feb. 24, 2022

(54) Fluoroscopic Tether Features to Indicate Proper Tether Tensioning

(71) Applicant: Tendyne Holdings, Inc., St. Paul, MN (US)

(72) Inventors: Preston James Huddleston, Maplewood, MN (US); Tracee Eidenschink, Wayzata, MN (US)

(21) Appl. No.: 17/405,593

(22) Filed: Aug. 18, 2021

Related U.S. Application Data
(60) Provisional application No. 63/067,557, filed on Aug. 19, 2020.

(51) Int. Cl. A61F 2/24

(52) U.S. Cl. CPC A61F 2/24; A61F 2220/0008; A61F 2210/0014; A61F 2250/0098

(57) ABSTRACT

A tether system for securing a prosthetic heart valve within a patient, the tether system comprising a tether defining a longitudinal axis, and a deformable element coupled to the tether, the deformable element having a first deformed shape in a first condition of the deformable element, and having a second substantially straight shape in a second condition of the deformable element so that, when the deformable element is in the second condition, the deformable element extends in a direction along the longitudinal axis.

### CROSS REFERENCE TO RELATED APPLICATIONS

The present application claims the benefit of the filing date of U.S. Provisional Patent Application No. 63/067,557, filed on Aug. 19, 2020, the disclosure of which is hereby incorporated herein by reference.

### BACKGROUND OF THE DISCLOSURE

The present disclosure relates to devices and methods for implanting a tethered prosthetic heart valve.

Various options are available to maintain a prosthetic heart valve in a desired position within a native heart valve annulus of a patient. For example, the position of a surgical prosthetic heart valve may be maintained by suturing the prosthetic heart valve into the patient's native heart valve annulus. Collapsible and expandable prosthetic heart valves, on the other hand, may be maintained in a desired position by exerting radial forces against the native heart valve annulus and/or surrounding tissue. It may additionally be beneficial to assist collapsible and expandable prosthetic heart valves in maintaining the desired position through the use of a tether that extends from the prosthetic heart valve to an anchor positioned in or on another structure of the heart, such as on an exterior portion of a ventricular wall of the patient's heart. However, during or after the implantation, it may be difficult to verify whether a sufficient or desired amount of tension has been placed on the tether to maintain the position of the prosthetic heart valve.

Thus, it would be preferable for a tether and prosthetic heart valve system to readily allow for verification that desired or sufficient tension has been placed on the tether.

### BRIEF SUMMARY

In accordance with one aspect of the disclosure, a tether system for securing a prosthetic heart valve within a patient, the tether system comprising a tether defining a longitudinal axis, and a deformable element coupled to the tether, the deformable element having a first deformed shape in a first condition of the deformable element, and having a second substantially straight shape in a second condition of the deformable element so that, when the deformable element is in the second condition, the deformable element extends in a direction along the longitudinal axis. The deformable element may be configured to transition from the first condition to the second condition upon application of a threshold tensile force to the tether. The deformable element may be configured to maintain the first deformed shape when a first tensile force is applied to the tether, the first tensile force being smaller than the threshold tensile force, and the deformable element is configured to maintain the second substantially straight shape when a second tensile force is applied to the tether, the second tensile force being equal to or greater than the threshold tensile force. The deformable element may be received within the tether and secured to the tether by one or more sutures. The first deformed shape may include a kink, a bend, or a curve. The deformable element may be one of radiopaque or echogenic, and formed of a shape memory material. The deformable element may be shape set so as to have the first deformed shape in the absence of applied force. The tether may include a first tether portion having an end coupled to a first end of the deformable element, and a second tether portion having an end coupled to a second end of the deformable element opposite the first end of the deformable element. When the deformed element has the first deformed shape, the deformed element may include a first deformity that extends along a first direction and a second deformity extending along a second direction, the first direction being transverse to the second direction. The tether system may further comprise a second deformable element coupled to the tether, the second deformable element having a first deformed shape in a first condition of the second deformable element, and having a second substantially straight shape in a second condition of the second deformable element so that, when the second deformable element is in the second condition, the second deformable element extends in the direction along the longitudinal axis. The first deformed shape of the first deformable element may include a first deformity and the second deformed shape of the second deformable element may include a second deformity, the first and second deformities extending along different directions.

In accordance with another aspect of the disclosure, a tether system for securing a prosthetic heart valve within a patient, the tether system comprising a tether, a deformable element coupled to the tether, and a tube received within the deformable element, the tether system having a first condition in which the tube has a first diameter along a length of the tube, the tether system having a second condition in which a central portion of the tube has a second diameter less than the first diameter, the deformable element having a third diameter along a length of the deformable element in the first condition of the tether system, and a central portion of the deformable element having a fourth diameter less than the third diameter in the second condition of the tether system. The tether system may be configured to transition from the first condition to the second condition upon application of a threshold tensile force to the tether. The tether system may be configured to be in the first condition when a first tensile force is applied to the tether, the first tensile force being smaller than the threshold tensile force, and the tether system may be configured to be in the second condition when a second tensile force is applied to the tether, the second tensile force being equal to or greater than the threshold tensile force. The central portion of the tube may include a pre-formed weak area. The pre-formed weak area may include at least one of (i) a decreased thickness relative to other portions of the tube, (ii) a greater elasticity relative to other portions of the tube, and (iii) a perforation. The deformable element may be one of radiopaque or echogenic, and formed of a shape memory material. The deformable element may be a braid. The suture may secure an end of the deformable element to the tether. The tether system may further comprise a clip positioned over the central portion of the tube, the clip having a first diameter in the first condition of the tether system and a second diameter in the second condition of the tether system, the second diameter being smaller than the first diameter. The central portion of the tube may include a detent. The tether system may further comprise a radiopaque ball positioned within the tether, wherein, in the first condition of the tether system, the ball is a spaced distance from the detent and, in the second condition of the tether system, the ball is received in the detent.

In accordance with another aspect of the disclosure, a tether system for securing a prosthetic heart valve within a patient, the tether system comprising a tether defining a longitudinal axis, a radiopaque ball received within the tether, and a tube surrounding a portion of the tether, the tube defining a first opening and a second opening, the second opening being positioned a spaced distance from the first opening along the longitudinal axis, wherein, in a first condition of the tether system, the radiopaque ball is received in the first opening and, in a second condition of the tether system, the radiopaque ball is received in the second opening. The tube further may comprise a slit wherein, in the first condition of the tether system, the slit is in an open condition and, in the second condition of the tether system, the slit is in a closed condition. The radiopaque ball may be secured to an end of a cable. The tether system may be configured to transition from the first condition to the second condition upon application of a threshold tensile force to the tether. The tether system may be configured to be in the first condition when a first tensile force is applied to the tether, the first tensile force being smaller than the threshold tensile force, and the tether system is configured to be in the second condition when a second tensile force is applied to the tether, the second tensile force being equal to or greater than the threshold tensile force. A suture may secure an end of the tube to the tether. In the first condition of the tether system, a diameter of a first end of the tube may be larger than a diameter of a second end of the tube and, in the second condition of the tether system, the diameter of the first end of the tube may be smaller than the diameter of the second end of the tube.

In accordance with another aspect of the disclosure, a method of tensioning a prosthetic heart valve system, the method comprising positioning a tether at least partially within a heart of the patient, the tether being coupled to at least one of a prosthetic heart valve and an anchor device, a deformable element being coupled to the tether and having a first deformed shape in the absence of applied force, tensioning the tether to a threshold tensile force and causing the deformable element, via the tensioning, to change into a second shape different than the first deformed shape, and imaging the deformable element to confirm if the deformable element has the first deformed shape or the second shape. Causing the deformable element to change into the second shape may include causing the deformable element to straighten so that the deformable element extends generally along a longitudinal axis of the tether. In the first shape of the deformable element, the deformable element includes a first deformity extending along a first direction and a second deformity extending along a second direction different from the first direction. Tensioning the tether may include tensioning a first portion of the tether coupled to a first end of the deformable element and a second portion of the tether coupled to a second end of the deformable element. A second deformable element may be coupled to the tether and has a third deformed shape in the absence of applied force, and tensioning the tether to the threshold tensile force may cause the second deformable element to change into a fourth shape different from the third deformed shape. The first deformed shape of the deformable element may include a first deformity extending along a first direction and the third deformed shape of the second deformable element may include a second deformity that extends along a second direction different form the first direction. During tensioning the tether, the tether may be fixed to one of the prosthetic heart valve and the anchor device, and after tensioning the tether, fixing the tether to the other of the prosthetic heart valve and the anchor device. The method may further comprise tensioning a tube received within the deformable element to the threshold tensile force to cause the tube to change from a third shape to a fourth deformed shape different from the third shape. A central portion of the tube may decrease in diameter as the tube changes from the third shape to the fourth deformed shape. As the deformable element changes from the first deformed shape to the second shape, a diameter of a portion of the deformable element may decrease. A clip may be positioned over the central portion of the tube, and as the central portion of the tube decreases in diameter, a diameter of the clip may also decrease. A radiopaque ball may be received within the tether, and tensioning the tether to the threshold tensile forces may cause the radiopaque ball to move from a first position relative to the tube to a second position relative to the tube. The radiopaque ball may be received within an opening or detent defined along the central portion of the tube when the radiopaque ball is in the second position.

In accordance with another aspect of the disclosure, a method of tensioning a prosthetic heart valve system, the method comprises positioning a tether at least partially within a heart of the patient, the tether being coupled to at least one of a prosthetic heart valve and an anchor device, and being received within a tube and defining a longitudinal axis, a radiopaque ball being positioned within the tether in a first position relative to the tube in the absence of applied forces, tensioning the tether to a threshold tensile force to move the radiopaque ball from the first position to a second position relative to the tube, the second position being a spaced distance from the first position along the longitudinal axis, and imaging the radiopaque ball to confirm if the radiopaque ball is at the first position or the second position. In the first position of the radiopaque ball, the radiopaque ball may be received in a first opening of the tube and, in the second position of the radiopaque ball, the radiopaque ball may be received in a second opening of the tube. The threshold tensile force may be about 3 lb. Tensioning the tether to the threshold tensile force may cause a slit defined along the tube to close. Tensioning the tether to the threshold tensile force may cause a diameter of a first end of the tube to decrease and a diameter of the second end of the tube to increase.

In accordance with another aspect of the disclosure, a tether system for securing a prosthetic heart valve within a patient, the tether system comprising a first tether portion and a second tether portion, a deformable element secured at a first end to the first tether portion and at a second end to the second tether portion, and the deformable element having a first diameter along a length of the deformable element in a first condition of the tether system, and a central portion of the deformable element having a second diameter less than the first diameter in a second condition of the tether system. The tether system may further comprise a ball received within the deformable element. The ball may have a diameter larger than the second diameter of the deformable element. The deformable element may have a thin section positioned between thick sections. In the second condition, the thick sections may have a third diameter and the thin section may have a fourth diameter less than the third diameter.

In accordance with another aspect of the disclosure, a tether system for securing a prosthetic heart valve within a patient, the tether system comprising a first tether portion and a second tether portion, a deformable element secured to the first tether portion, the deformable element defining a channel and a catch protruding within the channel, and an extension secured to the first tether portion, the extension positioned within the channel, and defining a first indent and a second indent, wherein, the catch of the deformable element engages the first indent in a first condition of the tether system and the catch of the deformable element engages the second indent in a second condition of the tether system. The extension may have a first diameter at a first end and a second diameter at a second end, the second diameter being greater than the first diameter. The deformable element may be a sleeve. The deformable element may include a plurality of fingers.

In accordance with another aspect of the disclosure, a tether system for securing a prosthetic heart valve within a patient, the tether system comprising a first tether portion and a second tether portion, a male coupling secured to the first tether portion, a shaft extending from the male coupling, and a female coupling secured to the second tether portion, the female coupling defining a first hole and a second hole, wherein, the shaft is received in the first hole in a first condition of the tether system and the shaft is received in the second hole in a second condition of the tether system.

### BRIEF DESCRIPTION OF THE DRAWINGS

These and other features, aspects, and advantages of the present disclosure will become better understood with regard to the following description, and accompanying drawings.

FIG. 1 is a cross-section of a portion of a heart with a prosthetic mitral valve implanted therein and an epicardial anchor device anchoring the mitral valve in position.

FIG. 2 is a schematic illustration of a prosthetic mitral valve and an epicardial anchor device.

FIG. 3 is a longitudinal cross-section of a tether system in a first deformed condition.

FIG. 4 is a longitudinal cross-section of the tether system of FIG. 3 in a second straightened condition.

FIG. 5 is a front view of a tether system in a first deformed condition, according to another aspect of the disclosure.

FIG. 6 is a side view of the tether system of FIG. 5 in the first deformed condition.

FIG. 7 is a side view of the tether system of FIG. 5 in a second straightened condition.

FIG. 8 is a front view of a tether system in a first deformed condition, according to another aspect of the disclosure.

FIG. 9 is a front view of the tether system of FIG. 8 in a second straightened condition.

FIG. 10 is a front view of a tether system in a first condition, according to an aspect of the disclosure.

FIG. 11 is an enlarged view of a portion of the tether system of FIG. 10 in a second condition.

FIG. 12 is a cross-sectional view of a tether system in a first condition along a first plane, according to an embodiment of the present disclosure.

FIG. 13 is a cross-sectional view of the tether system of FIG. 12 in the first condition along a second plane.

FIG. 14 is a cross-sectional view of a tether system, according to another aspect of the disclosure.

FIG. 15 is a side view of a tether system, according to another aspect of the disclosure.

FIG. 16 is a cross-sectional view of a tether system transitioning between a first condition and a second condition, according to an embodiment of the present disclosure.

FIG. 17 is a cross-sectional view of a tether system in a first condition, according to an embodiment of the present disclosure.

FIG. 18 is a cross-sectional view of the tether system of FIG. 17 in a second condition.

FIG. 19 is a side view of a tether system in a first condition, according to another aspect of the disclosure.

FIG. 20 is a side view of the tether system of FIG. 19 in a second condition.

FIG. 21 is a side view of a tether system in a first condition, according to another aspect of the disclosure.

FIG. 22 is a side view of the tether system of FIG. 21 in a second condition.

FIG. 23 is a side view of a tether system in a first condition, according to another aspect of the disclosure.

FIG. 24 is a side view of the tether system of FIG. 23 in a second condition.

FIG. 25 is a side view of a tether system in a first condition, according to another aspect of the disclosure.

FIG. 26 is a side view of the tether system of FIG. 25 in a second condition.

FIG. 27 is a side view of an extension, according to another aspect of the disclosure.

FIG. 28 is a side view of a tether system in a first condition, according to another aspect of the disclosure.

FIG. 29 is a side view of the tether system of FIG. 28 transition from the first condition to a second condition.

FIG. 30 is a side view of the tether system of FIG. 28 in the second condition.

FIG. 31 is a side view of a tether system prior to the male coupling engaging with the female coupling, according to another aspect of the disclosure.

FIG. 32 is a side view of the tether system of FIG. 31 in a first condition.

FIG. 33 is a side view of the tether system of FIG. 31 in a second condition.

FIG. 34 is a side view of a female coupling, according to another aspect of the disclosure.

### DETAILED DESCRIPTION

Some devices for anchoring a medical device, such as a collapsible and expandable prosthetic heart valve, can include securing one or more tethers extending from the medical device to an anchor positioned on the heart, such as along an exterior portion of the ventricular wall. In one exemplary valve replacement procedure, the prosthetic heart valve may be delivered to a native valve annulus while in a collapsed state, and then allowed to expand (either passively via self-expansion or actively via forced expansion) to at least partially secure the prosthetic heart valve within the native valve annulus. If the prosthetic heart valve is for use in replacing a native atrioventricular valve (i.e. mitral valve or tricuspid valve), a flared inflow end of the prosthetic heart valve may help prevent migration of the prosthetic heart valve into the ventricle, while a tether attached to the prosthetic heart valve may assist in preventing migration of the prosthetic heart valve into the atrium. During implantation, while the prosthetic heart valve is positioned within the valve annulus, a first end of the tether may be coupled to the prosthetic heart valve, and a second end of the tether may exit the heart, for example via a puncture in the left ventricular apex. While the second end of the tether is positioned outside the heart, an anchor may be slid over the tether until the anchor sits along the exterior portion of the ventricular wall such that an excess portion of the tether extends past the anchor outside the heart. The tether may then be tensioned to a desired force to maintain the position of the prosthetic heart valve within the patient's heart valve.

However, it may be difficult to verify that the tension is sufficient to maintain the position of the prosthetic heart valve. For example, although the pre-determined tension may be based on a standard force determined during testing, or based on the “feel” or intuition of the medical personnel during the implantation, such methods may require some level of intra-operative estimation as to whether the precise desired amount of tension has been placed on the tether. Thus, it may be beneficial to have a verification system to determine whether the tether has been placed under the desired amount of tension. This disclosure is directed to a tether system that enables an operator to visually verify, under imaging, that desired tension has been placed on a tether to assist in maintaining the position of the prosthetic heart valve.

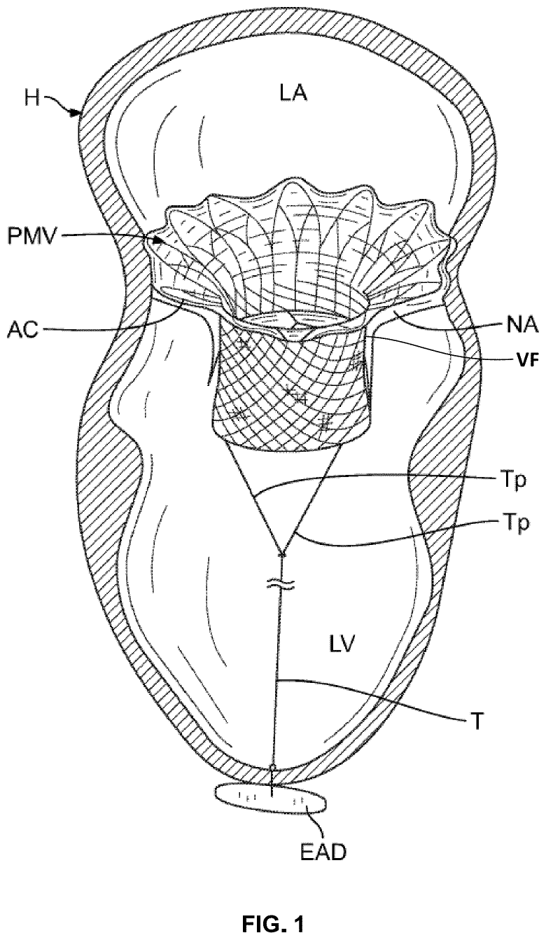

FIG. 1 is a cross-sectional illustration of the left ventricle LV and left atrium LA of a heart H having a transcatheter prosthetic mitral valve PMV deployed therein and an epicardial anchor device EAD securing the prosthetic mitral valve in place. FIG. 1 illustrates the prosthetic mitral valve PMV seated in the native annulus NA of the valve and held there using a valve frame VF of the prosthetic mitral valve, the radial tension from the native leaflets, and a ventricular tether T secured with attachment portions Tp to the prosthetic mitral valve and to the epicardial anchor EAD. An atrial flare portion, which may be referred to as an atrial cuff AC, of the valve frame VF may be positioned in the left atrium LA of the heart to prevent migration of the prosthetic mitral valve PMV into the left ventricle LV.

FIG. 2 is a schematic illustration of an epicardial anchor device 100 (also referred to herein as “anchor,” “anchor device,” or “epicardial anchor”) according to an embodiment of the disclosure. The anchor device 100 can be used to anchor or secure a prosthetic mitral valve PMV deployed between the left atrium LA and left ventricle LV of a heart. The anchor device 100 can be used, for example, to anchor or secure the prosthetic mitral valve PMV via a tether 128 as described above with respect to FIG. 1. The anchor device 100 can also seal a puncture formed in the ventricular wall (not shown in FIG. 2) of the heart during implantation of the prosthetic mitral valve PMV. The anchor device 100 can also be used in other applications to anchor a medical device (such as any prosthetic atrioventricular valve, including the tricuspid valve, or other heart valve) and/or to seal an opening such as a puncture.

The anchor device 100 can include a pad (or pad assembly) 120, a tether attachment member 124 and a locking pin 126. In some embodiments, the anchor device 100 can include a sleeve gasket (not shown in FIG. 2). The pad 120 can contact the epicardial surface of the heart and can be constructed of any suitable biocompatible surgical material. The pad 120 can be used to assist the sealing of a surgical puncture formed when implanting a prosthetic mitral valve. In some embodiments, the pad 120 can include a slot that extends radially to an edge of the pad such that the pad can be attached to, or disposed about, the tether 128 by sliding the pad onto the tether via the slot.

In some embodiments, the pad 120 can be made with a double velour material to promote ingrowth of the pad into the puncture site area. For example, pad or felt pledgets can be made of a felted polyester and may be cut to any suitable size or shape, such as those available from Bard® as PTFE Felt Pledgets having a nominal thickness of 2.87 mm. In some embodiments, the pad 120 can be larger in diameter than the tether attachment member 124. The pad 120 can have a circular or disk shape, or other suitable shapes.

The tether attachment member 124 can provide the anchoring and mounting platform to which one or more tethers 128 can be coupled (e.g., tied or pinned). The tether attachment member 124 can include a base member (not shown) that defines at least a portion of a tether passageway (not shown) through which the tether 128 can be received and pass through the tether attachment member, and a locking pin channel (not shown) through which the locking pin 126 can be received. The locking pin channel can be in fluid communication with the tether passageway such that when the locking pin 126 is disposed in the locking pin channel, the locking pin can contact or pierce the tether 128 as the tether passes through the tether passageway as described in more detail below with reference to specific embodiments.

The locking pin 126 can be used to hold the tether 128 in place after the anchor device 100 has been tightened against the ventricular wall and the tether has been pulled to a desired tension. For example, the tether 128 can extend through a hole in the pad 120, through a hole in a sleeve gasket (if the anchor device includes a sleeve gasket), and through the tether passageway of the tether attachment member 124. The locking pin 126 can be inserted or moved within the locking pin channel such that it pierces or otherwise engages the tether 128 as the tether extends through the tether passageway of the tether attachment member 124. Thus, the locking pin 126 can intersect the tether 128 and secure the tether to the tether attachment member 124.

The tether attachment member 124 can be formed with one or more of a variety of suitable biocompatible materials. For example, in some embodiments, the tether attachment member 124 can be made of polyethylene, or other hard or semi-hard polymer, and can be covered with a polyester velour to promote ingrowth. In other embodiments, the tether attachment member 124 can be made of metal, such as, for example, Nitinol, or ceramic materials. The tether attachment member 124 can be various sizes and/or shapes. For example, the tether attachment member 124 can be substantially disk shaped.

In some embodiments, the tether attachment member 124 can include a hub that is movably coupled to the base member of a tether attachment member. The hub can define a channel that can receive a portion of the locking pin (or locking pin assembly) 126 such that as the hub is rotated, the hub acts as a cam to move the locking pin 126 linearly within the locking pin channel. In this manner, the locking pin 126 is moved from a first position in which the locking pin is spaced from the tether passageway to a second position in which the locking pin intersects the tether passageway and engages or pierces a portion of the tether.

In use, after a prosthetic mitral valve PMV has been placed within a heart, the tether extending from the prosthetic mitral valve can be inserted into the tether passageway of the anchor device 100 and the tension on the tether attachment member 124 can be adjusted to a desired tension. Alternatively, in some cases, the tether extending from the prosthetic mitral valve PMV can be coupled to the anchor device 100 prior to the prosthetic mitral valve being placed within the heart. The anchor device 100 (e.g., some portion of the anchor device such as the tether attachment member 124, or a lever arm, or hub depending on the particular embodiment) can be actuated such that the locking pin 126 intersects the tether passageway and engages a portion of the tether 128 disposed within the tether passageway, securing the tether to the tether attachment member. In some embodiments, prior to inserting the tether 128 into the tether passageway, the anchor device 100 can be actuated to configure the anchor device to receive the tether. For example, if the tether attachment member 124 includes a lever arm movably coupled to the base member, the lever arm may need to be moved to an open position to allow the tether to be inserted into the tether passageway. Further details of epicardial anchor devices that may be suitable for use with the present disclosure are provided in U.S. Patent Application Publication Nos. 2016/0143736 and 2016/0367368, the disclosures of which are hereby incorporated by reference herein. Further details of prosthetic atrioventricular valves suitable for use with the present disclosure are provided in U.S. Patent Application Publication Nos. 2016/0074160 and 2017/0281343, the disclosures of which are hereby incorporated by reference herein.

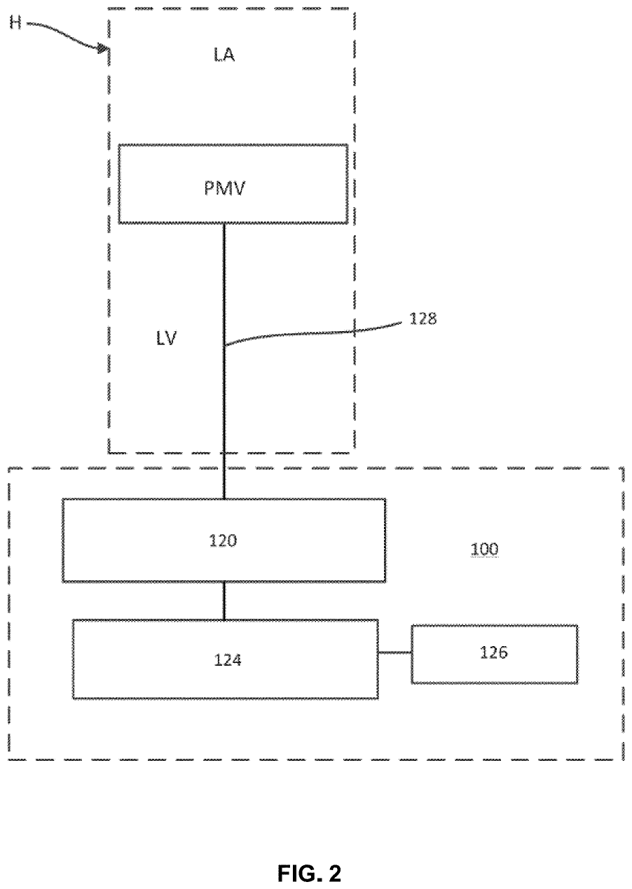

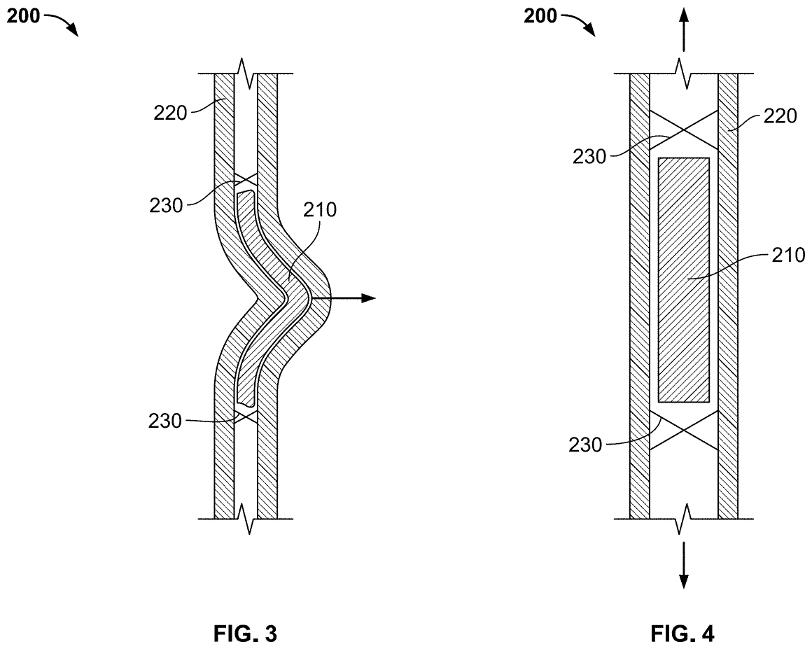

FIGS. 3-4 depict longitudinal cross-sections of a tether system 200 having a deformable element 210 received within tether 220. It should be understood that tether 220 may be used with a prosthetic heart valve and anchor device, including those described above and incorporated by reference. For example, although not illustrated in FIGS. 3-4, tether 220 may include a first end that is fixed to a frame of a prosthetic heart valve (either prior to, during, or after implantation of the prosthetic heart valve), and a second opposite end that is fixed to an anchor device anchored to a ventricular wall of the heart (either prior to, during, or after positioning of the anchor device on the heart). Element 210 is secured within tether 220 by sutures 230 such that the element is unable to translate within the tether along a longitudinal axis. Sutures 230 can be a knot, fabric, twist, or any other form of enclosure to axially secure deformable element 210. In an alternative embodiment, element 210 may be secured within tether 220 by a hook or staple. Sutures 230 can be made of the same material as tether 220. Alternatively, sutures 230 can be a stopping element installed within tether 220. In a further alternative, sutures 230 can be made of a separate material than the tether. It should be understood that structures other than sutures may be used to axially secure deformable element 210 within tether 220.

FIG. 3 depicts deformable element 210 secured by sutures 230 within tether 220 and in a first deformed condition with a deformity, such as a kink or bend, along a length of the element. Deformable element 210 may be formed so as to be in the first deformed condition in the absence of applied force, with the deformable element requiring a threshold amount of applied force to reduce or eliminate the deformity in the element. It should be understood that when the deformable element 210 is in the first deformed condition, the portion of tether 220 adjacent the deformable element may be in a corresponding deformed condition. As such, tether 220 and element 210 are in the first deformed condition when there is insufficient force applied to the element to straighten the element or to otherwise reduce or eliminate the deformity. This straightening force can be applied to element 210 through applying a longitudinal tension force to tether 220, indicated by the directional arrows shown in FIG. 4. For example, applying a longitudinal tension to tether 220 can pull the tether in a direction along an axis defined by the tether. This pulling force on tether 220 will tend to straighten the tether. Because the deformable element 210 is longitudinally held in place by sutures 230, the tensioning force applied to the tether 220 will tend to straighten the deformable element 210 in line with the axis of the tether, as shown in FIG. 4, as long as sufficient force is applied.

Deformable element 210 may be a wire designed such that a certain threshold amount of force is required to transition the deformable element 210 from the first deformed condition illustrated in FIG. 3 to a second straightened condition illustrated in FIG. 4. In one example, the deformable element 210 is formed as a tube or wire formed of a shape-memory material, such as a nickel titanium alloy, including Nitinol. The deformable element 210 may be shape set, for example via heat setting, so that the deformable element tends to revert to the first deformed condition in the absence of applied force, and requires a particular threshold tensile force to straighten out the deformable element 210. This threshold amount of force may be designed to correspond to the amount of tension on the tether 220 required to maintain the position of the prosthetic heart valve within the patient. For instance, tether 220 may require or otherwise be designed so that 3 lb of tension force is suitable to maintain the position of the prosthetic heart valve within the patient during normal pumping of the heart. In this example, element 210 may be shape set or otherwise designed to require 3 lb of tensile force to transition the deformable element to the second straightened condition from the first deformed condition. As such, when there is less than 3 lb of tensile force applied to tether 220 the deformable element 210 maintains at least some amount of the deformity (e.g. the bend or kink). As additional tensile force is applied to the tether 220, the deformable element 210 transitions to the second straightened condition. Preferably, upon application of the threshold tensile force to tether 220, the deformable element 210 is fully straightened, whereas the deformable element maintains a noticeable bend or kink when less than the threshold tensile force is applied to the tether. It should be understood that the threshold of 3 lb of tensile force described above is merely exemplary, and other threshold levels of tensile forces, including about 1 lb, about 2 lb, about 3 lb, about 4 lb, about 5 lb, or any other suitable amount of tensile force, may be suitable. It should be understood that these threshold levels of tensile forces may apply to all embodiments described herein.

Preferably, element 210 is made of a material that is capable of being visualized under fluoroscopy (e.g. a radiopaque, echogenic or fluroscopic material, or having a radiopaque marker), including metals or metal alloys such as Nitinol. In this manner, an operator may be able to visually verify whether element 210 is in a first deformed condition or second straightened condition. Because the condition of element 210 corresponds to whether there is sufficient tensile force applied to tether 220 to maintain the prosthetic heart valve within the patient, the surgeon can visually verify, under fluoroscopy or any other suitable imaging modality, that sufficient tension has been applied to the tether by confirming that the deformable element 210 is straight.

Although deformable element 210 is illustrated as being bent or kinked when in the first deformed condition, in alternative embodiments, the deformable element may be in the form of a curve, coil, disc, or the like when in the first deformed condition. In further alternatives, element 210 may have multiple deformities along the length of the element when in the first deformed condition. For instance, there may be multiple kinks or curves along the length of element 210 when in the first deformed condition. In a further alternative, there may be one or more markers placed along the length of element 210 to additionally assist in a surgeon verifying the tension of tether 220.

Even further, multiple deformities of element 210 may be positioned at different angles about a longitudinal axis of the tether. Such varying positions of the deformities can be beneficial where the deformity of element 210, such as a kink, is oriented towards the fluoroscopic equipment. For example, when the first deformed condition of element 210 includes a single kink and the kink extends along a direction directly towards or away from the fluoroscopic equipment, element 210 may appear, under imaging, as being in a second straightened condition even though the element is actually in a first bent or kinked condition.

With the above-described configuration, an operator may visually verify whether there is sufficient tension force applied to tether 220 from any imaging angle where element 210 has multiple deformities along its length and at least two of those deformities extend from the element about an axis of the tether in directions substantially transverse or orthogonal to each other. For example, element 210 may have a first kink extending along a first direction perpendicular to a longitudinal axis of tether 220 and a second kink extending along a second direction perpendicular to the longitudinal axis of the tether, and the second direction is at an angle transverse to the first direction about the longitudinal axis. In this manner, a surgeon may be able to visually verify whether element 210 is in its first or second condition when viewing the patient from any angle.

Tether 220 can be formed of any suitable material that allows visualization of the deformable element 210 within the tether. For example, tether 220 may be formed by a number of threads that are braided, woven, twisted, knitted, intermingled, or otherwise engaged to form a space within the tether to receive element 210. Tether 220 can be made from surgical-grade materials such as biocompatible polymer suture material. Examples of such material may include PTFE (polytetrafluoroethylene) or polypropylene. In one embodiment, tether 220 is inelastic. It is also contemplated that one or more of the tethers may optionally be elastic to provide an even further degree of compliance of the valve during the cardiac cycle.

As noted above, deformable element 210 may be constructed of a material that includes both heat-set properties and is capable of being visible in fluoroscopic imaging. Examples of such materials include nickel titanium alloys such as Nitinol and other shape memory materials, spring steel, polymers including BaSO₄ doped polymers, cobalt chromium, or other materials that are readily visible under fluoroscopy. In one embodiment, element 210 may be set to have a first shape at a first temperature (e.g., a temperature prior to being implanted within a patient) and a second shape at a second temperature (e.g., a temperature after being implanted within a patient). In this manner, prior to tether system 200 being implanted within the patient, element 210 may be set to have a shape that allows for easier and more efficient insertion within the patient, such as being straight. However, after prosthetic heart valve has been inserted within the patient, but before a sufficient tension force has been applied to tether 220 to overcome the rigidity of element 210, the element may be set to react to the temperature of the patient and transition to the first deformed condition.

Still referring to tether system 200 of FIGS. 3-4, it should be understood that, while the transition of the deformable element 210 from the first deformed condition to the second straightened condition may indicate that the desired threshold tension has been applied to tether 220, it may not indicate whether the tether 220 has been over tensioned. In other words, if deformable element 210 is set to straighten upon application of 3 lb of tensile force, and only 1 lb of tensile force is applied to the tether 220, it will be clear under imaging that too little tensile force has been applied. On the other hand, if 5 lb of tensile force is applied to the tether 220, the deformable element 210 will have straightened, but it may not be obvious under imaging that the tether 220 may have been over-tensioned. It should be understood that the tether 220 is under greater tension during ventricular systole when the ventricle is pumping, since the prosthetic valve is closed and resisting pressure from the pumping ventricle. On the other hand, during ventricular diastole when the ventricle is filling, the tether 220 is under a smaller amount of tension since the prosthetic heart valve is open to allow blood to flow from the atrium to the ventricle. It would thus be preferable to set the threshold force to transition from the first deformed condition to the second straightened condition at a level which is equal to or greater than tension experienced by the tether 220 during ventricular systole, but less than the tension experienced by the tether during ventricular diastole. With this configuration, the deformable element 210 may switch between the first deformed condition and the second straightened condition, and back again, as the ventricle cycles between ventricular systole and ventricular diastole, respectively. Thus, the operator can confirm that the desired tension has been reached, while also confirming that the tether has not been over-tensioned. If the tether 220 has been over-tensioned, it may always be in the second straightened condition, without transitioning back to the first deformed condition when tension on the tether is reduced during ventricular diastole. It should be understood that this feature may apply to some or all embodiments described herein.

Still further, it should be understood that a prosthetic heart valve that is secured to the heart via a tether may experience a change in loads over time. For example, even if a prosthetic heart valve is implanted with an ideal tension on the tether, as time progresses, tension on the tether may increase or decrease, for example as a result of anatomical changes, as a result in slight positional changes of the prosthetic heart valve and/or anchor, or as a result of the tether itself relaxing. Because the deformable element 210 remains visible under fluoroscopy, it is possible to non-invasively visualize the deformable element 210 days, weeks, or months after the implantation procedure to confirm whether the tension on the tether 220 is still in the desired range. If it is determined that tension on the tether 220 is undesirable, it may be possible to perform a corrective procedure based on the information learned from the imaging of the deformable element 210. It should be understood that this feature may apply to some or all embodiments described herein.

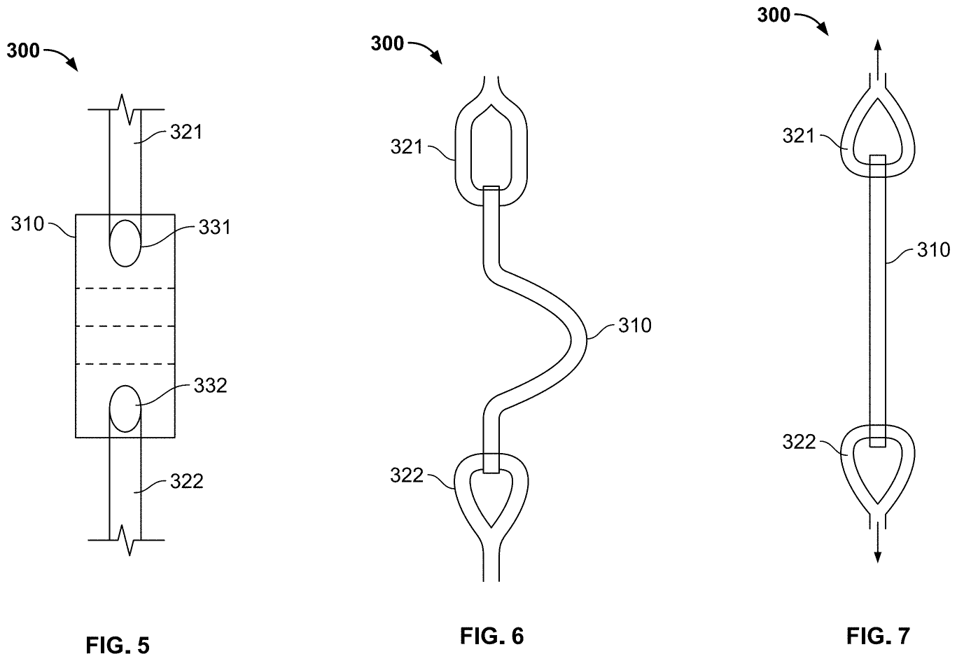

In an alternative embodiment, the element may be a helical spring or a flat spring. For example, FIGS. 5-7 depict tether system 300 including features similar or identical to those of tether system 200, except as discussed below. FIGS. 5-6 depict deformable element 310 in a first deformed condition. Element 310 includes multiple bends along the length of the element, and includes a first hole 331 adjacent a first end of the element and a second hole 332 adjacent a second end of the element. The tether may be formed as two tether portions, including a first tether portion 321 that forms a knot, braid, weave, twist, knit, or the like through first hole 331 to secure the first end of element 310, and a second tether portion 322 similarly secured through second hole 332 to secure the second end of the element. Thus, in this embodiment, one of the first tether portion 321 and the second tether portion 322 secures the prosthetic valve to the deformable element 310, and the other of the first tether portion 321 and the second tether portion 322 secures the epicardial anchor to the deformable element 310.

Deformable element 310 may be designed to require a certain amount of tensile force to transition from the first deformed condition to the second straightened condition in substantially the same manner as described above in connection to deformable element 210. FIG. 7 depicts tether portions 321, 322 having been tensioned in opposite longitudinal directions to apply the threshold tensile force to transition the deformable element 310 into the straightened condition.

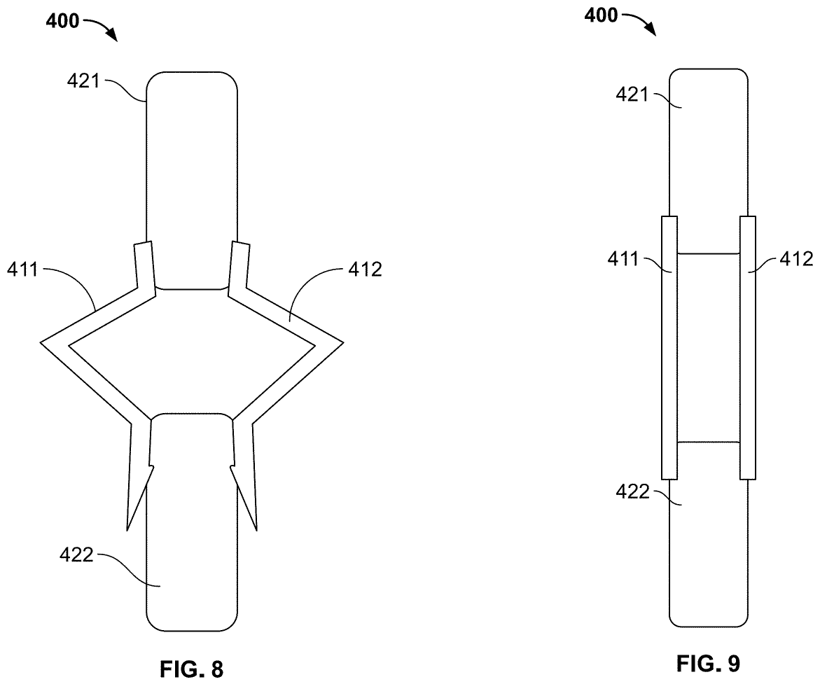

In some embodiments, more than one element may be engaged to the tether. For example, FIGS. 8-9 depict a tether system 400 including features similar or identical to those of tether systems 200, 300, except that there are two deformable elements 411, 412. In the first deformed condition of the deformable elements, deformable element 411 has a bend or kink that is oriented in a direction away from deformable element 412, and vice versa, such that the bend or kink of both elements extend along different directions. Such opposing deformable elements 411, 412 may enable an operator to more easily and efficiently visually verify whether sufficient tension has been applied to the tether portions 421, 422. Each end of each deformable element 411, 412 may be attached to corresponding ends of the tether portions 421, 422 in any suitable fashion, including by suturing, adhesives, bonding, stapling, hooks, etc. Although tether system 400 is illustrated with a single pair of deformable elements 411, 412 on substantially diametrically opposite sides of the tether portions 421, 422, it should be understood that a second pair of deformable elements may be included, so that the tether system 400 includes a total of four deformable elements spaced at about 90 degree intervals around the diameter of the tether portions 421, 422. The inclusion of four deformable portions may increase the likelihood that deformation may be readily visible in any orientation or imaging plane, and may also provide additional stability in connecting the two tether portions 421, 422. However, it should be understood that any desirable number of deformable elements may be included in tether system 400 and each deformable element may be spaced about the diameter of the tether portions at any interval angles.

One embodiment of the use of tether system 200 will now be described with reference to FIGS. 3-4. However, it is understood that a similar method may be applied to tether systems 300, 400.

After the prosthetic heart valve has been seated within the native annulus, with the tether 220 extending out of the apex of the heart, tether 220 is tensioned such that the operator believes there is the desired amount of tension to maintain the position of the prosthetic heart valve during the cardiac cycle of the patient. As an example, this may be set to an initial predetermined tension that is measured by a tensioning device used to tension the tether or may be determined by the operator as being sufficient tension by the operator's intuition or “feel.” However, it would be beneficial for the operator to verify that the desired level of tension has been achieved. Whether this verification is done post-operatively or intra-operatively, the operator may examine the patient using a fluoroscopy to determine the straightened or deformed condition of element 210.

For example, the operator may view that element 210 is in a deformed condition as shown in FIG. 3 while in ventricular diastole, and therefore lacking sufficient tension. The surgeon may then increase the tension on tether 220, such as through a tensioning instrument (not shown) engaged to the apical pad (as depicted in FIG. 1). As the surgeon applies additional tensile force to tether 220, element 210 will tend to straighten as sutures 230 prevent longitudinal movement of the element within the tether. As such, tensioning tether 220 can change the position of element 210 from a first condition where the element is deformed to a second condition where the element is straightened once the surgeon has applied a threshold amount of tensile force. Where the threshold amount of tensile force is the same or similar amount of tensile force required to maintain the position of the prosthetic heart valve, the surgeon can visually verify that element 210 is in a straightened condition during ventricular systole while having a deformed condition during ventricular diastole to confirm that there is sufficient tension of tether 220 to maintain the position of the prosthetic heart valve, but that the tether is not over-tensioned either. Methods of use of tether systems 300 and 400 are substantially similar or identical to that of tether system 200.

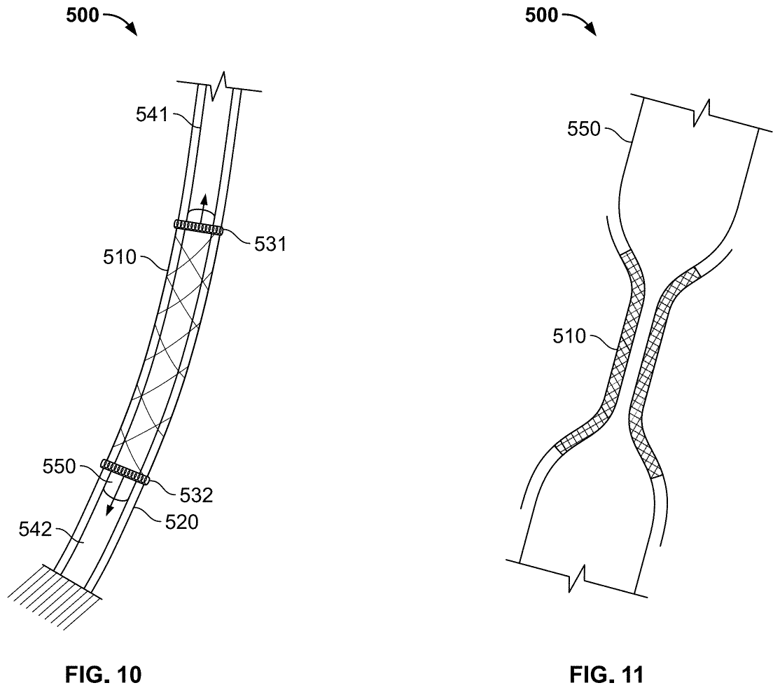

FIGS. 10-11 depict tether system 500 including many of the same or similar features of tether systems 200, 300, 400, with certain exceptions described below. Deformable element 510 may take the form of a collapsible tube, braid or similar structure secured at a first end to an exterior surface of tether 520 via any suitable mechanism, such as a suture 531, and at a second end via any suitable mechanism, such as a suture 532. Alternatively, deformable element 510 may be secured to an interior surface of tether 520. In one alternative, element 510 may be sutured (or otherwise fixed) with only one of suture 531 or suture 532 to enable movement of the element relative to tether 520. Element 510 may be formed of a shape memory material configured to constrict down to a resting diameter without any force being applied to the element, and a material that is readily visible under fluoroscopy (e.g., Nitinol or the like). Alternatively or additionally, element 510 may be configured to constrict down onto a tube 550 only after the element enters the patient's body and is heated up by the body temperature of the patient.

Tube 550 is attached to a first cable portion 541 at a first end and to a second cable portion 542 at a second end. Tube 550 and cable portions 541, 542 are received within tether 520 such that tensioning the cables (and, by extension, the tube) also tensions the tether, and vice versa. Second cable portion 542 may be coupled to either the prosthetic heart valve or the epicardial anchor, with first cable portion 541 being free so that it may be pulled to increase tension on tube 550. Tube 550 can be made of a material, such as polyethylene terephthalate (PET) or the like, such that a portion of the tube deforms from a first condition having a relatively large first diameter to a second deformed condition having a second relatively smaller diameter after a threshold amount of tensile force is applied the tube. The deformable element 510 may be shape-set to have an unbiased diameter that is smaller than the first diameter of the tube 550. However, the rigidity of tube 550 may prevent the deformable element 510 from transitioning to its smaller, shape-set unbiased diameter.

As a threshold amount of tensile force is applied to tube 550, a central portion of the tube begins to decrease in diameter from the first relatively large diameter to the second relatively small diameter to form a second condition having a “neck,” as depicted in FIG. 11. In the second condition, the diameter of element 510 also forms a “neck” as the element may be able to constrict down (partially or entirely) to its smaller unbiased shape-set diameter. In some instances this unbiased shape-set diameter of element 510 may correspond to the necking condition of tube 550 as the tube is tensioned. Alternatively, this unbiased shape-set diameter may be larger than the second diameter of the necking condition of tube 550.

This necking condition may be further enabled by weak points or areas along a central portion of tube 550 to allow for the tube to more readily transition from the first relatively large diameter condition to the second relatively small diameter necking condition. Such weak points may include a perforation, detent, slit, thinner material, or the like. Alternatively or additionally, the central portion of tube 550 may be constructed of a more malleable or elastic material than other portions of the tube.

In one example, as depicted in FIG. 10, tether system 500 is in a first condition after being implanted within the patient prior to a sufficient tensile force applied to tether 520, cable 541, 542, and tube 550 to deform the tube. In this first condition, the element 510 is unable to transition to its smaller unbiased diameter because tube 550 has enough rigidity to prevent element 510 from collapsing to its smaller unbiased diameter. Tube 550 is preferably formed with a rigidity (or other structural characteristics) such that the threshold amount of tensile force (e.g., 3 lb of force) required to deform the tube into the necking condition (and thus allow element 510 to similarly deform into a necking condition) is similar or identical to the desired tensile force on tether 520 to assist in maintaining the position of the prosthetic heart valve.

FIG. 11 depicts tether system 500 in a second condition after a sufficient tensile force greater than the threshold tensile force is applied to tether 520 and cable 541, 542 to cause necking of the tube 550. It should be understood that the tether 520 is omitted from the view of FIG. 11. In this second condition, the tension applied to tether 520 and cables 541, 542 pulls the tube 550 along an axis of the tether so as to form a necking condition, thereby allowing element 510 to begin to transition to its smaller unbiased diameter, conform to the tube, and also form a necking condition. Tether system 500 can transition from the first generally cylindrical condition to the second deformed or necking condition in ventricular systole, and back to the first generally cylindrical condition in ventricular diastole. Because the deformable element 510 is readily visible under fluoroscopy, a surgeon can visually verify the threshold tension of tether 520 has been reached by verifying the necking condition of element 510 during ventricular systole Similarly, the surgeon can visually verify that the tether 520 has not been over-tensioned by visually verifying the transition back to the generally cylindrical condition of element 510 during ventricular diastole.

In a method of use of tether system 500, tensioning tether 520 and cables 541, 542 can cause tube 550 to “neck down” or have a necking condition. Tube 550 can be configured to have a necking condition corresponding a sufficient amount of tension to maintain the position of the prosthetic heart valve during ventricular systole. Where element 510 is configured to have a smaller resting diameter than the diameter tube 550 in a first condition, as depicted in FIG. 10, the necking condition of the tube allows the element to correspondingly minimize in diameter and undergo a necking condition similar to the tube, as depicted in FIG. 11. The surgeon can then visually verify that sufficient tension has been applied to tether 520 during ventricular systole by determining whether element 510 has a necking condition.

In an alternative embodiment, the cables 541, 542 may be omitted, and tube 550 may be sewn within element 510 to be held longitudinally in place by sutures 531, 532 similar to element 210 between sutures 230 as depicted in FIGS. 3-4. In this manner, the operation may involve less equipment and/or complexity.

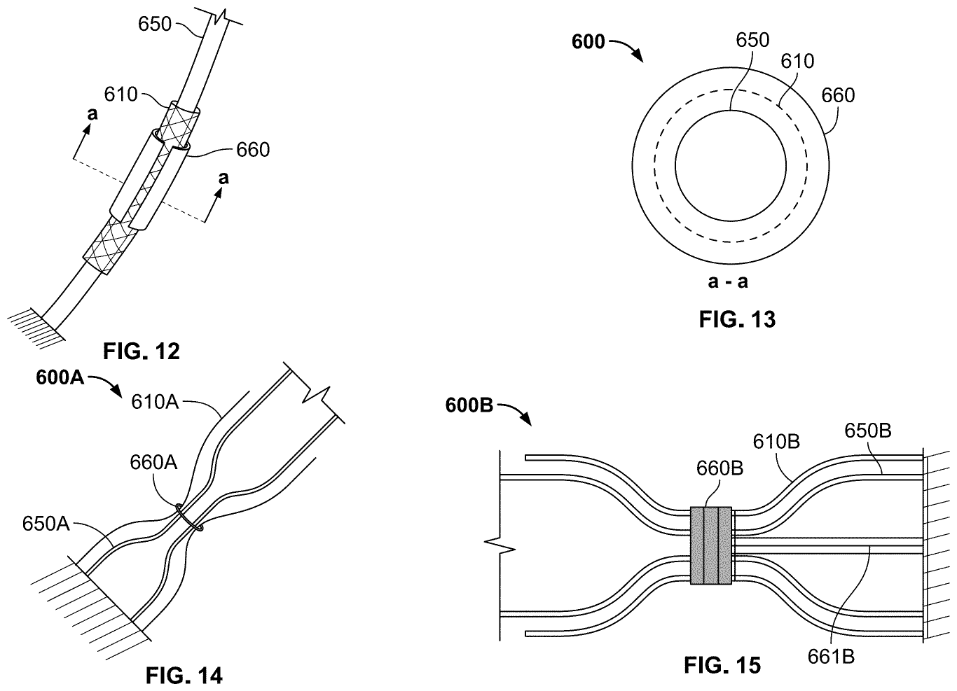

The tether system may additionally include a fluoroscopic, echogenic, or radiopaque ring that can provide additional visual indication of whether sufficient tension is applied to the tethers. For example, FIGS. 12-13 depict a schematic view of tether system 600 in the process of being tensioned including the features of tether system, 500, with certain exceptions as discussed below. Clip 660 (which may alternatively be referred to as a locking ring) partially encircles an exterior surface of deformable element 610, which may be similar or identical to deformable element 510. Clip 660 may be formed of a shape-memory or spring-type material, and set to have a first resting diameter less than a first diameter of tube 650 in a first cylindrical condition. In this manner, as tube 650 (and thus deformable element 610) decreases in diameter to the second necked condition, the clip 660 will also transition to a smaller diameter.

In this manner, clip 660 is in its first relatively large diameter condition when insufficient tensile force has been applied to tube 650 to induce the tube to enter a necking condition and, therefore, to induce element 610 to enter a corresponding necking condition, as shown in FIGS. 12-13. However, when a sufficient tensile force has been applied to element 610 and to tube 650 to transition the element and tube to enter the necking condition, clip 660 may transition to its second relatively smaller diameter less than the first diameter of the clip. For instance, in the second diameter of clip 660, the ends of the clip may contact each other so as to completely encircle the element and tube. Alternatively, the second diameter of clip 660 may not completely encircle around the element and tube in the second condition but, instead, simply has a smaller diameter than the first resting diameter of the clip in the first condition. In this manner, clip 660 can provide a further visual indication that sufficient tension has been applied to the tether by highlighting the necking condition of tube 650.

Alternatively or additionally, the clip 660 may have a spring or other biasing force to maintain the tube 650 and deformable element 610 in the second necked condition. For instance, a first end of clip 660 may be received into a second end of clip 660 and prevented from disengagement. With this locking functionality, clip 660 may help to provide a clear indication that the tube 650 and the deformable element 610 have been tensioned at least to the desired threshold tension of the tether. In other words, clip 660 may provide an irreversible indication that at least the threshold tension has been reached.

Alternatively, clip 660 may be a knot or band wrapped around the tube and deformable element. For example, FIG. 14 depicts a tether system 600A similar or identical to those of tether system 600 except, instead of a clip, the tether system includes a knot or band that encircles tube 650A and deformable element 610A. Tie 660A may be made of a radiopaque or echogenic elastic material. In this manner, as tube 650A (and thus deformable element 610A) decreases in diameter to the necked condition, the tie 660A will also transition to a smaller diameter.

In a method of use of tether system 600, the method may be similar to tether system 500 except clip 660 is included to provide additional visual verification of the tension of the tether system. Clip 660 will have a first diameter in a first condition, prior to tube 650 and element 610 undergoing a necking condition, that is larger than a second diameter in a second condition, after the tube and element undergo the necking condition. In one example, changing from the first diameter to the second diameter may include both ends of clip 660 contacting each other. In this manner, clip 660 can highlight the necking condition of element 610 and provide additional visual verification that sufficient tension has been applied to tether 620 during ventricular systole. However, it is understood that a similar method may be applied to tether system 600A.

In a further alternative, tie 660A may be a knot secured to a cable. For example, FIG. 15 depicts a tether system 600B similar or identical to those of tether system 600A except, instead of a band or clip, the tether system may include a knot 660B tied around tube 650B and deformable element 610B. Knot 660B is secured at a first end of a cable 661B. Cable 661B may be secured at a second end to either an epicardial anchor or a prosthetic heart valve along an exterior surface of deformable element 610B. Knot 660B can be tied to have a slip knot or noose-like configuration with cable 661B such that, as a tensile force is applied to the cable, the knot can tighten around tube 650B and deformable element 610B. In this manner, as tube 650A and thus deformable element 610A decreases in diameter to the necked condition, the tie 660A will also transition to a smaller diameter.

In a method of use of tether system 600B, the method may be similar to tether system 500, 600, 600A except knot 660B is included to provide additional visual verification of the tension of the tether system. Knot 660B will have a first diameter in a first condition, prior to tube 650B and deformable element 610B undergoing a necking condition, that is larger than a second diameter in a second condition, after the tube and element enters the necking condition.

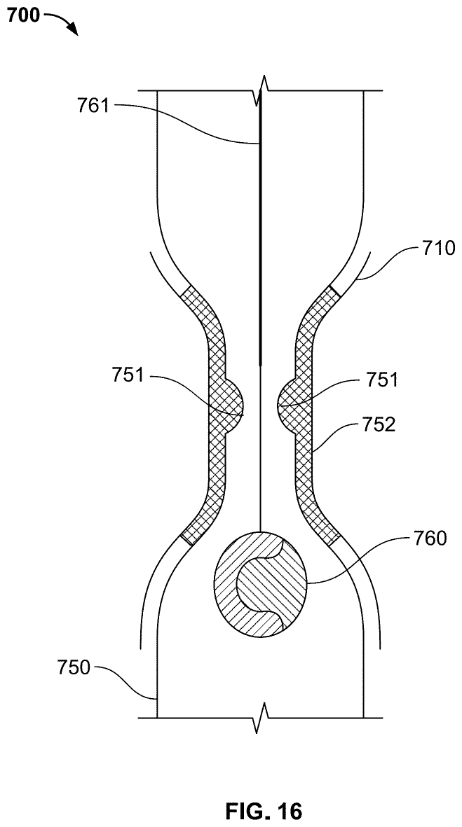

In another embodiment, the tethering system may include a radiopaque or echogenic rigid mass, such as a ball, that provides further visual verification of the tension of the tether system. FIG. 16 depicts tether system 700 in the process of being tensioned and including many similar or identical features of tether systems 200, 300, 400, 500, 600, with certain exceptions described below. Ball 760 is secured to a first end of cable 761, which may be a suture, while the second end of the cable is attached to either a prosthetic heart valve or epicardial anchor device, as described above in connection with FIG. 2. Ball 760 can be formed of a radiopaque or echogenic material, including any biocompatible metal, such that the ball can be readily visualized during under fluoroscopy.

Tube 750 can be secured at one of a first or second end to a tether (not shown) and can be made of a substantially rigid material, such as stainless steel or the like. Tube 750 has a neck region 752 having a smaller diameter than a diameter adjacent the ends of the tube. Neck region 752 defines detents 751 sized to receive ball 760. Ball 760 has a diameter that is greater than the diameter of neck region 752 of tube 750 such that, prior to a sufficient amount of tensile force being applied to the tether secured to tube 750, the ball is unable to enter the neck region. Detents or openings 751 may be defined along a portion of tube 750 and may be sized to receive ball 760 when a tensile force is applied to the tether secured to tube 750. Specifically, detents 751 receive ball 760 when a precise amount of tensile force is applied to the tether secured to tube 750, such as the precise amount of tension required to maintain the position of the prosthetic heart valve without over-tensioning tether system 700. The operator may then visually verify under fluoroscopy whether there is adequate tension being placed on the prosthetic heart valve by whether ball 760 is received in detents 751.

Sleeve 710 may be secured to a tether (not shown) at one of a first or second end. Sleeve 710 can be made of a radiopaque or echogenic material having a different level of radiodensity to ball 760. In this manner, sleeve 710 may provide a visual contrast to ball 760 to assist an operator in verifying the tension of cable 761. In other embodiments, there may be no sleeve 710. In a further alternative, tube 750 may alternatively be secured within a single tether connecting the epicardial anchor to the prosthetic heart valve.

Although two detents 751 are depicted in FIG. 16, in alternative embodiments there may be any number of detents, such as three or more. Further, there may be multiple sets of detents longitudinally spaced along tube 750 with each set of detents having a different diameter. For instance, a first set of detents 751 can be defined along a first diameter of tube 750 and a second set of detents can be defined along a second diameter of the tube, where the second diameter is greater than the first diameter. In this manner, an operator may be able to more accurately determine the level of tensile force applied to the tether secured to tube 750 as a first tensile force may be required to pull the cable into the first set of detents 751 and a second tensile force to pull the cable into the second set of detents.

In a method of use of tether system 700 the method may be similar to tether system 500 except ball 760 is additionally included to provide visual verification of the tension of the tether system. Ball 760 may be received within a tether (not shown) prior to sufficient tension being applied to the tether secured to tube 750. However, once a precise amount of tensile force has been applied to tether system 700, and specifically to the tether secured to tube 750, to cause the tube to slide over ball 760 such that the ball may be received in detents 751 within the neck region 752. In this manner, the ball being received within the detents may provide additional visual verification that a precise amount of tension has been applied to tether system 700 without over-tensioning the tether.

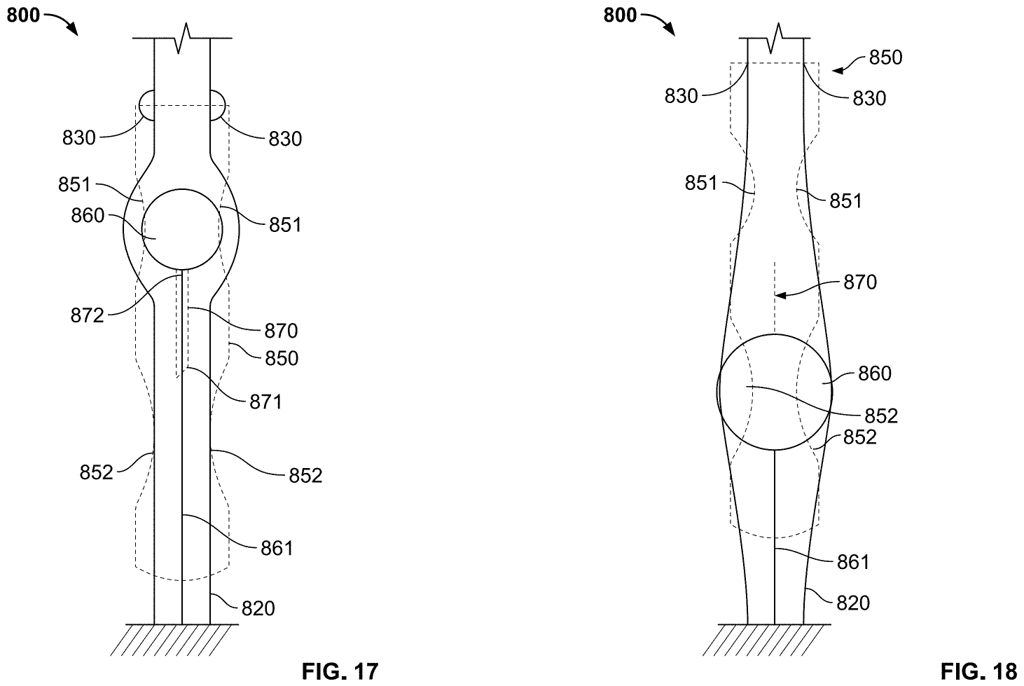

FIGS. 17-18 depict tether system 800 including features similar or identical to those of tether systems 200, 300, 400, 500, 600, 700, with certain exceptions described below. In this embodiment, a radiopaque or echogenic ball 860 is received within tether 820, with the ball 860 attached to either the prosthetic valve or the epicardial anchor via a cable 861, which may be for example a suture.

Tube 850 is secured over an exterior portion of tether 820 through sutures 830 or other suitable mechanism. Tube 850 may be made of a material that is readily visible under fluoroscopy such as Nitinol or stainless steel. A slit 870 may be formed along a length of the tube 850 and may be open or closed depending on whether a radial expansion force is applied to a first end 871 or second end 872 of the slit. Slit 870 can achieve this open or closed condition by having a triangular shape such that a first end 871 of the slit converges to a point while a second end 872 opposite the first end is cut to have a larger width than the first end. In this manner, when ball 860 is received within first detents 851, the slit 870 may define an opening or gap along a portion of the tube 850. On the other hand, when ball 860 is received within second detents 852, the outward radial force applied on the tube 850 by the ball may cause the slit 870 to close. This configuration of slit 870 provides greater control of the tensile force applied to tube 850 as the tube transitions from a first condition to a second condition.

FIG. 17 depicts tether system 800 in a first condition, prior to sufficient tensile force being applied to tether 820. In this first condition, ball 860 is at a first position received within detents 851. While ball 860 is at the first position, ball 860 distends the portions of tether 820 in contact with the ball, which also distends the portions of tube 850 adjacent detents 851 and second end 872 of slit 870 causing the slit to open. In this manner, a surgeon may determine that there is insufficient tension applied to tether 820 through one or both of the position of ball 860 being received in detents 851, or slit 870 having an opened condition.

FIG. 18 depicts the tether system in a second condition, after sufficient tensile force has been applied to tether 820. In the view of FIG. 18, the tether 820 is pulled in the upward direction to tension the tether 820. As the tether 820 begins to tension, both the tether 820 and the attached tube 850 move a distance in the upward direction relative to ball 860, which has a substantially fixed position due to its connection via cable 861 to either the prosthetic valve or the epicardial anchor. As the tube 850 translates relative to ball 860, the ball exits the first detents 851 and translates relative to tube 850 until it is received within the second detents 852. While ball 860 is at this second position, the ball distends the portions of tether 820 in contact with the ball, which also distends the portions of tube 850 adjacent detents 852 and first end 871 of slit 870. As there is no distension force of tube 850 adjacent second end 872, the slit is now in a closed position. In this manner, a surgeon may determine that there is sufficient tension applied to tether 820 through one or both of the position of ball 860 being received in detents 852, or slit 870 having a closed condition.

Alternatively, there may be any number of slits 870 along tube 850, such as no slits or more than one slit. In a further alternative, each end of tube 850 may be differently sized such that one end is larger than the other. For example, in the first condition, a first end of tube 850 can be larger than a second end of the tube while, in the second condition, the second end of the tube can be larger than the first end of the tube. In a yet further alternative, tether system 800 may have only one of ball 860 and cable 861, or slit 870 to provide visual verification of the tension of tether system.

Turning to tether system 800 depicted in FIGS. 17-18, tensioning tether 820 translates the position of ball 860 relative to the tether and tube 850 from a first condition where the ball is at a first position received within detents 851 and slit 870 has an open position to a second condition, where the ball is at a second position received within detents 852 and the slit has a closed condition. Ball 860 may be received in detents 852 once the tensile force applied to tether 820 is sufficient to maintain the position of the prosthetic heart valve. The surgeon can then visually verify that sufficient tension has been applied to the tether.

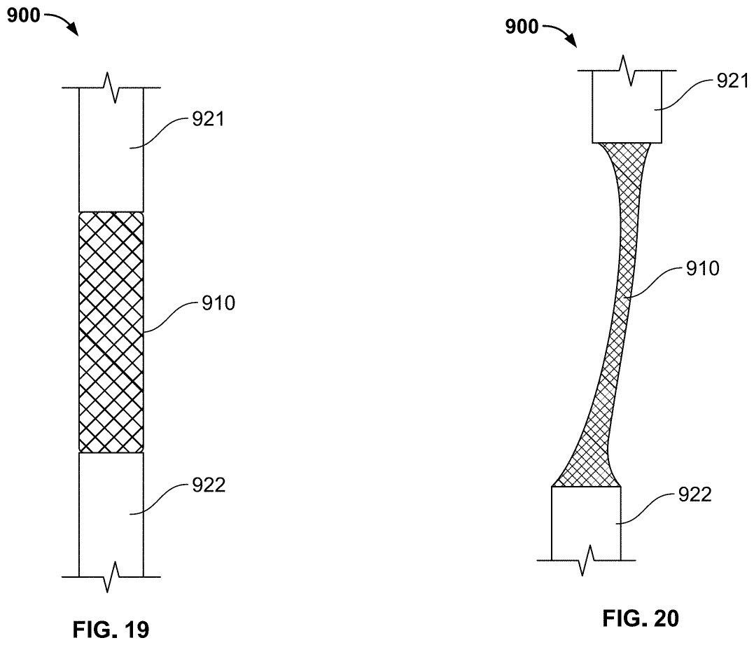

In another example, there may be two tether portions similar to tether system 400 depicted in FIGS. 8-9, however there may only be one deformable element secured between the two tether portions. FIGS. 19-20 depict another example tether system 900 including features similar or identical to those of tether system 200, 300, 400, 500, 600, 700, 800, 900 with certain exceptions described below. Each end of deformable element 910 is secured to respective ends of tether portions 921, 922. Deformable element 910 can be a braid, collapsible tube, or the like. Element 910 can deform from a first, unbiased condition having a relatively large first diameter to a second, deformed condition having a second relatively smaller diameter after a threshold amount of tensile force is applied the element. In this manner, as a threshold amount of tensile force is applied to element 910, a central portion of the element begins to decrease in diameter from the first relatively large diameter to the second relatively small diameter to form a second condition having a “neck,” as depicted in FIG. 20.

Alternatively, a first condition of element 910 may have a first condition having a spherical or ellipsoid shape with a first diameter, and a second condition having a second, smaller diameter with either a substantially cylindrical shape or necking condition when tether portions 921, 922 is tensioned. In this manner, an operator can verify whether sufficient tension has been applied to tether portions 921, 922 when viewing the deformable element from any angle.

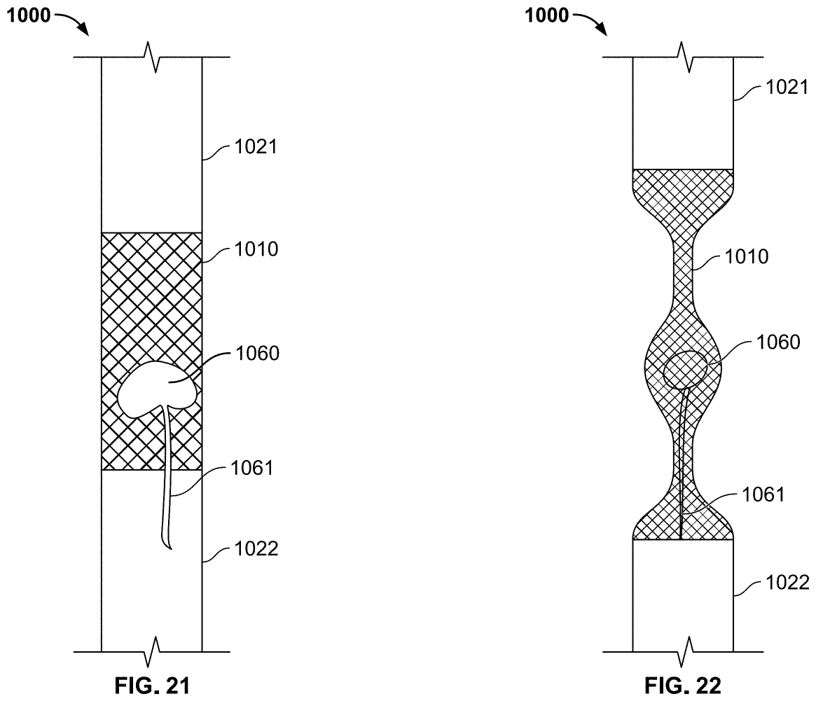

In another example, there may be both a deformable element and ball in the tether system. FIGS. 21-22 depict another example tether system 1000 including features similar or identical to those of tether systems 200, 300, 400, 500, 600, 700, 800, 900 with certain exceptions described below. Each end of deformable element 1010 is secured to respective ends of tether portions 1021, 1022. Ball 1060 is secured to a first end of cable 1061 while a second end of the cable is attached to either a prosthetic heart valve or epicardial anchor device. Element 1010 can be a braid, collapsible tube, or the like. Element 1010 deforms from a first, unbiased condition having a relatively large first diameter to a second, deformed condition having a second relatively smaller diameter after a threshold amount of tensile force is applied to the element. One or both of deformable element 1010 and ball 1060 can be formed of a radiopaque or echogenic material. In the deformed condition, a portion of element 1010, such as the central portion of the element, can conform to the shape of ball 1060. In this manner, an operator can verify whether sufficient tension has been applied to tether portions 1021, 1022 by detecting whether element 1010 displays a bulbous protrusion along a length of the element.

Where element 1010 is a substantially semi-rigid material, deformation of element 1010 from the first condition to the second condition may result in the element permanently maintaining its deformed condition around the ball. In this manner, where the tension of tether portions 1021, 1022 is released, ball 1060 will remain retained within the element. Alternatively, where element 1010 is a shape-memory material, such as nitinol, the element may revert back to its first condition from the second condition, thus releasing ball 1060.

In an alternative embodiment, there may be more than one ball 1060, such as two, three, or the like. Having multiple balls 1060 can provide a clearer indication that there is sufficient tension applied on tether portions 1021, 1022. Further, each ball 1060 may have a different size corresponding to a certain tensile force such that element 1010 deforming about a first ball having a first diameter might indicate that, although tension is being applied to tether portions 1021, 1022, more tension is required until the tether portions deform about a second ball having a second diameter less than the first diameter, thus indicating that the prosthetic heart valve has been sufficiently tensioned.

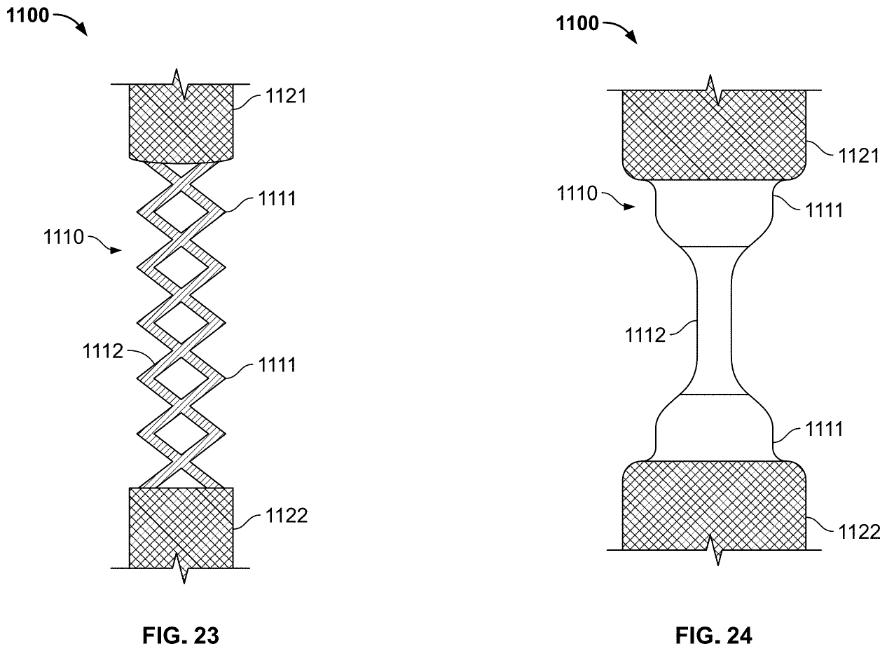

In another example, the deformable element may include a thin and thick section. FIGS. 23-24 depict another example tether system 1100 including features similar or identical to those of tether systems 200, 300, 400, 500, 600, 700, 800, 900, 1000 with certain exceptions described below. Deformable element 1110 is secured to respective ends of tether portions 1121, 1122. Element 1110 is a braid, collapsible tube, or the like having thick sections 1111 and a thin section 1112 positioned between the thick sections. The walls of element 1110 along thick section 1111 has a greater thickness than the walls of the element along the thin section 1112. In this manner, when element 1110 transitions from a first, unbiased condition to a second, deformed condition, thin section 1112 will decrease in diameter prior to thick section 1111. Moreover, when element 1110 is in the deformed condition, thin section 1112 has a smaller diameter than thick section 1111. In this manner, an operator can visually verify that tension is being applied to tether portions 1121, 1122 from thin section 1112 entering a necking condition but that more tension may be required until thick section 1111 enters a deformed condition, thus indicating that the prosthetic heart valve is sufficiently tensioned. In an alternative embodiment, there may be more than two sections of varying thicknesses, such as three, four, or the like. In a further alternative, thick section 1111 can be a section of element 1110 where an extra layer of material is overlaid on thin section 1112 such that the total thickness of the walls of the thick section is greater than that of the thin section.

In a method of use of tether system 900, tether portions 921, 922 may be tensioned from a first condition where element 910 has a first diameter to a second condition where the element has a second diameter smaller than the first diameter. Such tensioning may require that element 910 enters a necking condition can require a sufficient amount of tensile force being applied to the tether portions. It is understood that a similar method may be applied to tether system 1000, 1100.

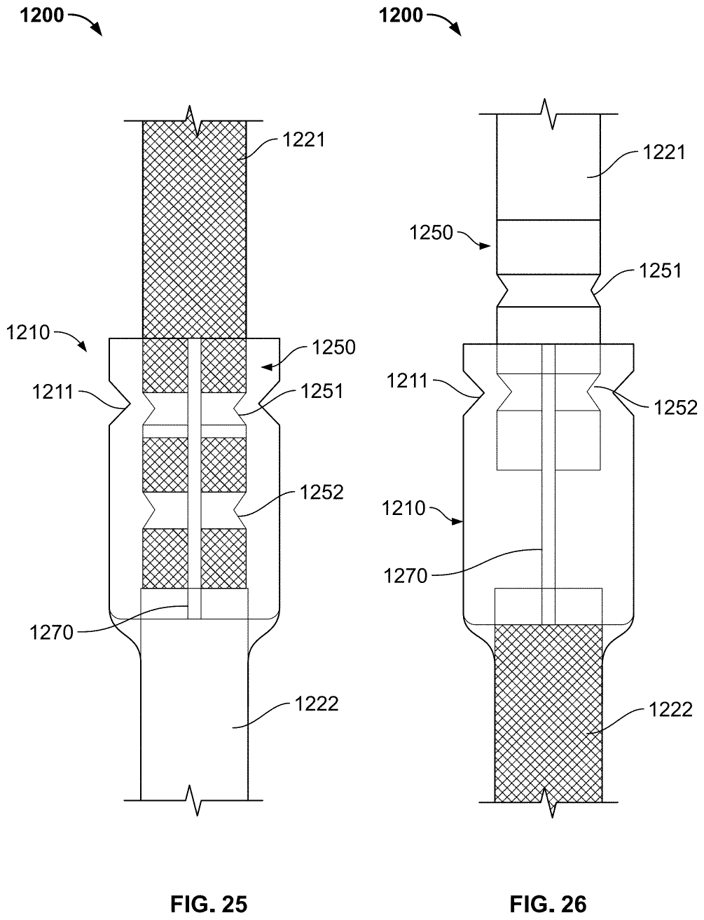

In another example, the deformable element may be a sleeve engaged with an extension engaged to a tether portion. FIGS. 25-26 depict another example tether system 1200 including features similar or identical to those of tether systems 200, 300, 400, 500, 600, 700, 800, 900, 1000, 1100 with certain exceptions described below. Tether system 1200 includes sleeve 1210 engaged with extension 1250. Sleeve 1210 is secured at an end to a corresponding end of tether portion 1222. Sleeve 1210 is substantially cylindrical and defines a channel therethrough. Sleeve 1210 includes a catch 1211 radially and circumferentially protruding inwardly within the sleeve. Sleeve 1210 defines an indent along an exterior surface corresponding to the interior protrusion of catch 1211 within the sleeve. In an alternative embodiment, sleeve 1210 may be substantially smooth without any indents defined along the exterior surface. Sleeve 1210 defines a slit 1270 along the length of the sleeve. Slit 1270 can allow for sleeve 1210 to open along the slit when a radial force is internally applied to the sleeve.

Extension 1250 is secured at an end to a corresponding end of tether portion 1221 and is received within the channel of sleeve 1210. Extension 1250 is substantially cylindrical and circumferentially defines a first indent 1251 and a second indent 1252 at a longitudinal distance away from the first indent. First indent 1251 can be configured to receive catch 1211 such that a first amount of tensile force is required to be applied to sleeve 1210 to dislodge the catch from the first indent. Second indent 1252 can have a similar configuration as the first indent. However, in an alternative embodiment, second indent 1252 can be configured to receive catch 1211 such that a second tensile force required to dislodge the catch from the second indent, the second tensile force being greater than the first tensile force. For instance, first indent 1251 can have rounded edges such that catch 1211 can more easily slide over and dislodge from the first indent while second indent 1252 can have angular edges such that it can be more difficult to dislodge the catch from the second indent. Alternatively, extension 1250 can have a different thickness at each of indents 1251, 1252 that require a different amount of tensile force being applied to sleeve 1210 to overcome.

In a first condition, prior to sufficient tension being applied to tether portions 1221, 1222, catch 1211 can be received within first indents 1251. The amount of tensile force required to dislodge catch 1211 from first indent 1251 may correspond to a desired amount of tensile force sufficient to tension the prosthetic heart valve. In this manner, when the desired amount of tensile force applied to tether portions 1221, 1222 is reached, catch 1211 may be dislodged from first indent 1251 such that sleeve 1210 opens up along slit 1270 and slides along an exterior surface of extension 1250 until the catch is received within second indent 1252, in a second condition. The desired amount of tensile force may be less than the amount of tensile force required to dislodge catch 1211 from second indent 1252 so that there is less risk of sleeve 1210 from disengaging completely with extension 1250. As such, an operator may visually verify whether sufficient tension has been applied to tether portions 1221, 1222 by whether catch 1211 is received within first indent 1251 or second indent 1252.

Sleeve 1210 can be a first radiopaque or echogenic material while extension 1250 can be a second radiopaque or echogenic material. For example, extension 1250 can be a polymer, such as an acrylonitrile butadiene styrene (ABS) polymer, or the like, while sleeve 1210 can be a shape-memory material, such as Nitinol, or the like. In this manner, an operator can visually compare the engagement of sleeve 1210 with respect to extension 1250.

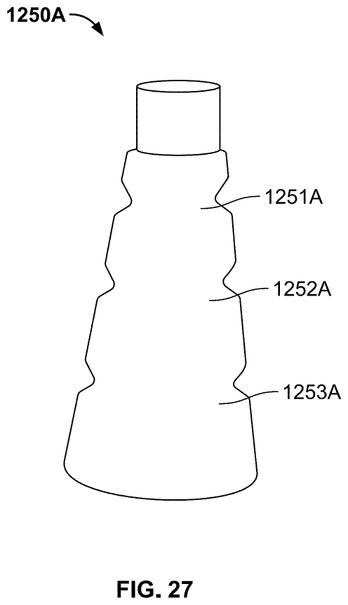

In a further alternative, the extension may have any more than two indents. For example, FIG. 27 depicts extension 1250A having a number of indents 1251A, 1252A, 1253A. Extension 1250A can have a different width or thickness at each of indents 1251A, 1252A, 1253A to increase the amount of tensile force required to be applied to the tether portions (not shown) to overcome the engagement between a catch of a sleeve (not shown) and indents. For instance, a tensile force of 1.5 lbs may be required to overcome the engagement between the catch and indent 1251A, 3 lbs may be required to overcome the engagement between the catch and indent 1252A, and 5 lbs lbs may be required to overcome the engagement between the catch and indent 1253A.

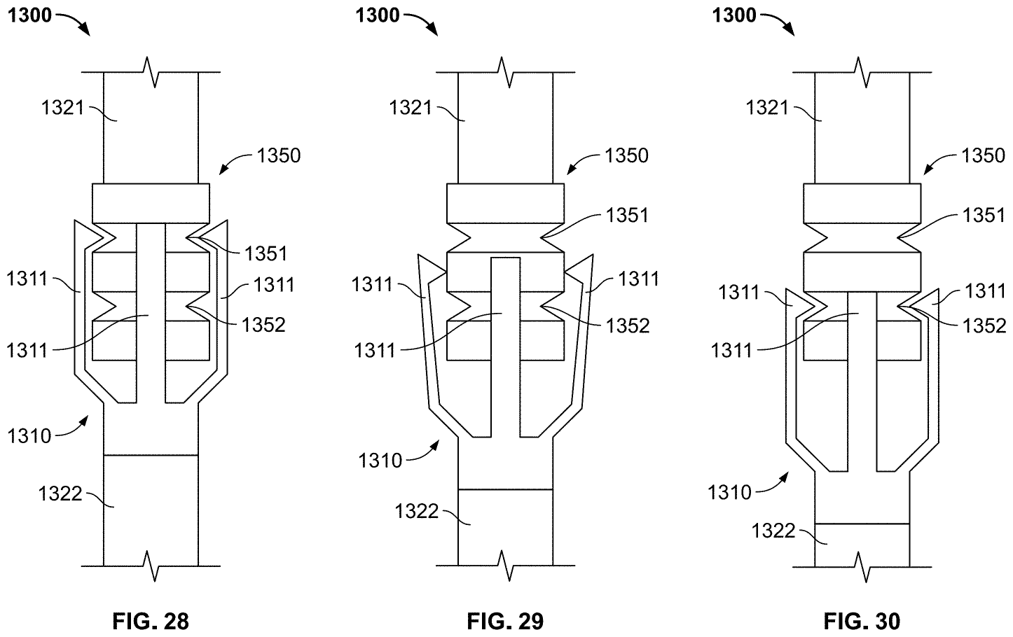

In an alternative embodiment, the sleeve and the catch may not circumferentially enclose the extension and, instead, the sleeve may be a number of fingers engaging the extension. FIGS. 28-30 depicts tether system 1300 including features similar or identical to those of tether systems 200, 300, 400, 500, 600, 700, 800, 900, 1000, 1100, 1200 with certain exceptions described below. In this embodiment, grip 1310 engages extension 1350. Grip 1310 includes a plurality of fingers 1311 that are hooked at its end. In this manner, in a first condition, fingers 1311 can engage first indent 1351 and, in a second condition, fingers 1311 can engage second indent 1352. In transitioning from the first condition to the second condition, fingers 1311 can expand about a base point of grip 1310 adjacent tether portion 1322 or along any portion of the fingers. Although three fingers 1311 are depicted, in alternative examples, there may be any number of fingers, such as two, four, five, or the like. In an alternative embodiment, indents 1351, 1352 may not be circumferential and be distinct detents configured to receive each of fingers 1311.

In a method of use of tether system 1200, tether portions 1221, 1222 may be tensioned from a first condition where catch 1211 is received in first indent 1251 to a second condition where the catch is received in second indent 1252. Such tensioning may require that a sufficient tensile force be applied to the tether portions to dislodge the catch from the first indent. This tensile force may be a similar tensile force to sufficiently tension the prosthetic heart valve. Once catch 1211 is dislodged from first indent 1251, sleeve 1210 expands by opening up along slit 1270 to allow the sleeve to slide over extension 1250 until catch 1211 is received in second indent 1252. It is understood that a similar method may be applied to tether system 1300 and a tether system having extension 1250A.

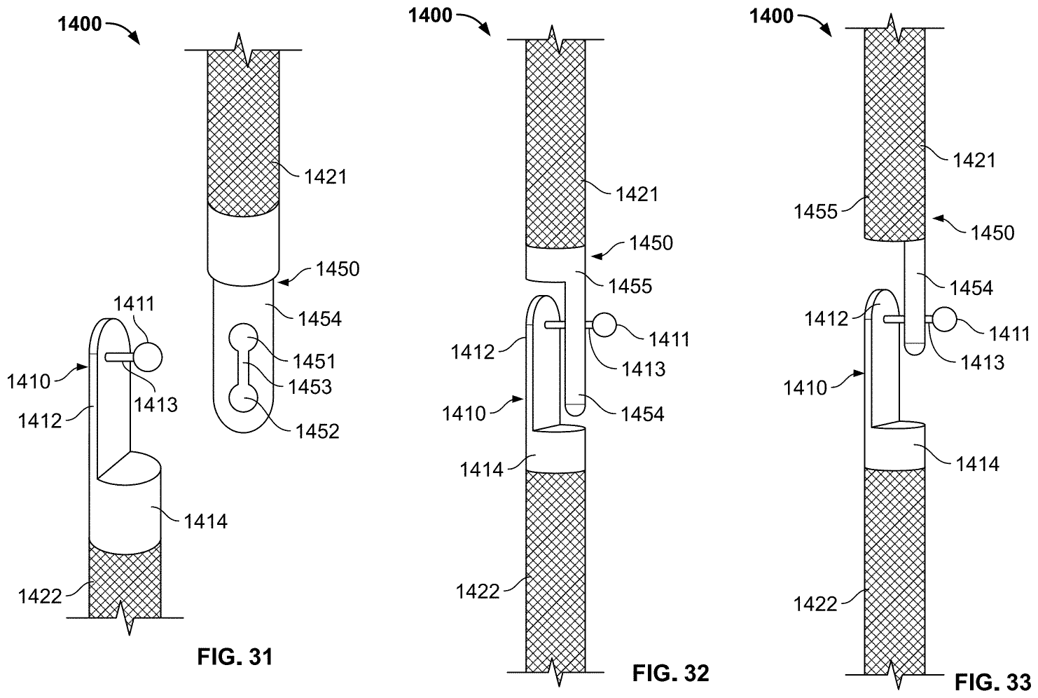

In a further alternative, the tether portions may be tensioned through a male and female coupling system. FIGS. 31-33 depicts tether system 1400 including features similar or identical to those of tether systems 200, 300, 400, 500, 600, 700, 800, 900, 1000, 1100, 1200, 1300 with certain exceptions described below. Tether system 1400 includes a male coupling 1410 secured at an end to a corresponding end of tether portion 1422 and a female coupling 1450 secured at an end to a corresponding end of tether portion 1421. One or both of male coupling 1410 and female coupling 1450 can be a radiopaque or echogenic and/or shape-memory material as described above.

Male coupling 1410 includes a male base 1414 directly secured at a first end to tether portion 1422 and a male body 1412 extending from a second end of the base along a longitudinal axis of the male coupling. Male base 1414 has a circumference substantially similar to the circumference of tether portion 1422. Male body 1412 has a width defined by a partial circumference about the diameter of the male base. In this manner, male coupling 1410 defines a space between male body 1412 and male base 1414 such that the male coupling has a substantially L-shape when viewed from a side profile.

Male coupling 1410 includes a pin shaft 1413 extending from male body 1412 at a transverse angle from the longitudinal axis of the male coupling. Pin shaft 1413 includes a pin head 1411 secured at an end of the pin shaft. Pin head 1411 is substantially spherical however, in other examples, the pin head may have any other shape, such as cuboid, pyramidal, or the like. Pin head 1411 has a diameter larger than pin shaft 1413.

Female coupling 1450 has a female base 1455 and female body 1454 similar to male base 1414 and male body 1412 except, rather than pin shaft 1413 and pin head 1411, female coupling 1450 defines a first hole 1451, a second hole 1452 being a longitudinal distance from the first hole, and a slit 1453 defined between the first hole and second hole. First hole 1451 and second hole 1452 each has a diameter sized to receive pin head 1411 and pin shaft 1413 of male coupling 1410. Slit 1453 has a width smaller than the diameter pin shaft 1413.

In this manner, in a first condition, prior to a sufficient tension being applied to tether portions 1421, 1422, pin shaft 1413 and pin head 1411 are received within first hole 1451 and male coupling 1410 cannot be longitudinally displaced within slit 1453 without a further tensile force being applied to the tether portions to push the pin shaft into the slit. The ratio of the width between slit 1453 and pin shaft 1413 may be configured such that the amount of force required to pull the pin shaft into the slit is the same amount of tensile force required to optimally tension the prosthetic heart valve. In this first condition, there may be no gap defined between a tip of female body 1454 and male base 1414, and a tip of male body 1412 and female base 1455.

Once this tensile force is applied to tether portions 1421, 1422, pin shaft 1413 can enter slit 1453 to translate within the slit until the pin shaft is received within second hole 1452, in a second condition of tether system 1400. In this second condition, an operator may be able to verify that sufficient tension has been applied by determining the existence of a gap defined between at least one of a tip of female body 1454 and male base 1414, or a tip of male body 1412 and female base 1455. In an alternative embodiment, there may be a first gap defined in a first condition and a second gap defined in a second condition, where the second gap is larger than the first gap.

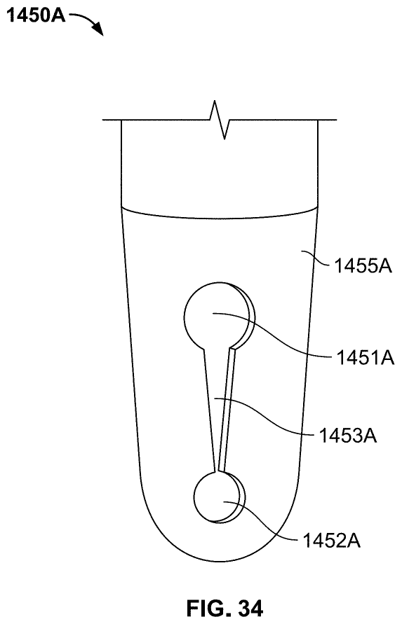

In alternative embodiments, base 1414, 1455 may have a larger or smaller diameter than tether portions 1422, 1421. In a further alternative, female coupling 1450 can taper in width from first hole 1451 to second hole 1452. FIG. 34 depicts female coupling 1450A having a slot 1453A tapering from a larger width adjacent first hole 1451A to a smaller width adjacent second hole 1452A. In this manner, a greater tensile force is needed as a pin shaft (not shown) translates within slot 1453A from first hole 1451A to second hole 1452A. In a further alternative, slot 1453A can have a smaller width adjacent first hole 1451A and a larger width adjacent second hole 1452A.

In a method of use of tether system 1400, pin head 1411 and pin shaft 1413 may first be inserted within first hole 1451 such that the tether system is in a first condition, prior to sufficient tension being applied to tether portions 1421, 1422. A tensile force can be applied to tether portions 1421, 1422 until a sufficient tension is reached such that pin shaft 1413 enters slit 1453. Pin shaft 1413 slides along slit 1453 until second hole 1452 is reached and tether system 1400 enters a second condition. It is understood that a similar method may be applied to tether system having female coupling 1450A.

The tether systems and prosthetic heart valves of this disclosure can be implanted in a transapical or transseptal approach. In transapical delivery, a small incision is made between the ribs and into the apex of left ventricle to deliver the prosthetic heart valve to the target site. In a transapical approach, the free end of the tether is positioned outside the heart and is pulled in order to tension the tether, and the tension may be locked via any suitable mechanism, such as through the use of a pinning mechanism within an epicardial anchor while the free end of the tether extends through a center for the epicardial anchor. In the embodiments described above that include a radiopaque ball, the radiopaque ball is coupled to the prosthetic valve via a cable, suture, or the like, if a transapical approach is used. In a transseptal approach of implanting a prosthetic heart valve, the prosthetic heart valve is passed through the septum between the right atrium and left atrium. In a transseptal approach, the tether is typically fixedly connected to the epicardial anchor, and prosthetic heart valve is slid over a free end of the tether. The tether is tensioned by pulling the tether, and the tether is locked to the prosthetic heart valve via any suitable mechanism, such as barbs or a crimping tool, once the tether has achieved the desired tension. In the embodiments described above that include a radiopaque ball, the radiopaque ball is coupled to the epicardial anchor via a cable, suture, or the like, if a transseptal approach is used.

Although many embodiments above are described in connection with a physical deformation of a tether (or a component operatively associated with the tether) that is viewable under fluoroscopy or another imaging modality to determine tension on the tether, other modalities may be used to achieve this goal. For example, any suitable mechanism may be applied so that, when the tether is under a first tension, the tether (or the component operatively associated therewith) has a first configuration, and when under a second tension greater than the first tension, the tether (or the component operatively associated therewith) has a second configuration distinct from the first configuration. The difference between the first configuration and the second configuration may be any change that is capable of being sensed. For example, the tether may include a piezoelectric component so that, upon experiencing load, the piezoelectric component changes configuration, resulting in a generation of an electric charge that may be identified by any suitable sensing mechanism. In other embodiments, the tether may include chamber with two or more chemicals separate by a frangible wall or other mechanism. In yet other embodiments, tension may cause a temperature change that may be viewed with thermal imaging. Upon experiencing a predetermined load, the frangible wall (or other separation mechanism) may break and allow the two or more chemical to mix, resulting in an output that may be sensed to indicate the change in configuration. Such an output may be a change in phosphorescence detectable by any suitable means. In other words, the change in configuration in the tether (or associated component) following application of load to the tether may be provided by any indicia suitable for detection following implantation.

According to one aspect of the disclosure, a tether system for securing a prosthetic heart valve within a patient, the tether system comprises:

a tether defining a longitudinal axis; and

a deformable element coupled to the tether, the deformable element having a first deformed shape in a first condition of the deformable element, and having a second substantially straight shape in a second condition of the deformable element so that, when the deformable element is in the second condition, the deformable element extends in a direction along the longitudinal axis; and/or

the deformable element is configured to transition from the first condition to the second condition upon application of a threshold tensile force to the tether; and/or

the deformable element is configured to maintain the first deformed shape when a first tensile force is applied to the tether, the first tensile force being smaller than the threshold tensile force, and the deformable element is configured to maintain the second substantially straight shape when a second tensile force is applied to the tether, the second tensile force being equal to or greater than the threshold tensile force; and/or

the deformable element is received within the tether and secured to the tether by one or more sutures; and/or

the first deformed shape includes a kink, a bend, or a curve; and/or

the deformable element is radiopaque and formed of a shape memory material; and/or

the deformable element is shape set so as to have the first deformed shape in the absence of applied force; and/or

the tether includes a first tether portion having an end coupled to a first end of the deformable element, and a second tether portion having an end coupled to a second end of the deformable element opposite the first end of the deformable element; and/or

the deformed element has the first deformed shape, the deformed element includes a first deformity that extends along a first direction and a second deformity extending along a second direction, the first direction being transverse to the second direction; and/or

the tether system includes a second deformable element coupled to the tether, the second deformable element having a first deformed shape in a first condition of the second deformable element, and having a second substantially straight shape in a second condition of the second deformable element so that, when the second deformable element is in the second condition, the second deformable element extends in the direction along the longitudinal axis; and/or

the first deformed shape of the first deformable element includes a first deformity and the second deformed shape of the second deformable element includes a second deformity, the first and second deformities extending along different directions.

According to another aspect of the disclosure, a tether system for securing a prosthetic heart valve within a patient, the tether system comprises:

a tether;

a deformable element coupled to the tether; and

a tube received within the deformable element, the tether system having a first condition in which the tube has a first diameter along a length of the tube, the tether system having a second condition in which a central portion of the tube has a second diameter less than the first diameter, the deformable element having a third diameter along a length of the deformable element in the first condition of the tether system, and a central portion of the deformable element having a fourth diameter less than the third diameter in the second condition of the tether system; and/or

the tether system is configured to transition from the first condition to the second condition upon application of a threshold tensile force to the tether; and/or

the tether system is configured to be in the first condition when a first tensile force is applied to the tether, the first tensile force being smaller than the threshold tensile force, and the tether system is configured to be in the second condition when a second tensile force is applied to the tether, the second tensile force being equal to or greater than the threshold tensile force; and/or

the central portion of the tube includes a pre-formed weak area; and/or

the pre-formed weak area includes at least one of (i) a decreased thickness relative to other portions of the tube, (ii) a greater elasticity relative to other portions of the tube, and (iii) a perforation; and/or

the deformable element is radiopaque and formed of a shape memory material; and/or

the deformable element is a braid; and/or

a suture secures an end of the deformable element to the tether; and/or

the tether system includes a clip positioned over the central portion of the tube, the clip having a first diameter in the first condition of the tether system and a second diameter in the second condition of the tether system, the second diameter being smaller than the first diameter; and/or

the central portion of the tube includes a detent; and/or

the tether system includes a radiopaque ball positioned within the tether, wherein, in the first condition of the tether system, the ball is a spaced distance from the detent and, in the second condition of the tether system, the ball is received in the detent.

According to another aspect of the disclosure, a tether system for securing a prosthetic heart valve within a patient, the tether system comprises:

a tether defining a longitudinal axis;

a radiopaque ball received within the tether; and

a tube surrounding a portion of the tether, the tube defining a first opening and a second opening, the second opening being positioned a spaced distance from the first opening along the longitudinal axis,

wherein, in a first condition of the tether system, the radiopaque ball is received in the first opening and, in a second condition of the tether system, the radiopaque ball is received in the second opening; and/or

the tube includes a slit wherein, in the first condition of the tether system, the slit is in an open condition and, in the second condition of the tether system, the slit is in a closed condition; and/or

the radiopaque ball is secured to an end of a cable; and/or

the tether system is configured to transition from the first condition to the second condition upon application of a threshold tensile force to the tether; and/or

the tether system is configured to be in the first condition when a first tensile force is applied to the tether, the first tensile force being smaller than the threshold tensile force, and the tether system is configured to be in the second condition when a second tensile force is applied to the tether, the second tensile force being equal to or greater than the threshold tensile force; and/or

a suture secures an end of the tube to the tether; and/or

in the first condition of the tether system, a diameter of a first end of the tube is larger than a diameter of a second end of the tube and, in the second condition of the tether system, the diameter of the first end of the tube is smaller than the diameter of the second end of the tube.

According to another aspect of the disclosure, a method of tensioning a prosthetic heart valve system, the method comprises:

positioning a tether at least partially within a heart of the patient, the tether being coupled to at least one of a prosthetic heart valve and an anchor device, a deformable element being coupled to the tether and having a first deformed shape in the absence of applied force;

tensioning the tether to a threshold tensile force and causing the deformable element, via the tensioning, to change into a second shape different than the first deformed shape; and

imaging the deformable element to confirm if the deformable element has the first deformed shape or the second shape; and/or

causing the deformable element to change into the second shape includes causing the deformable element to straighten so that the deformable element extends generally along a longitudinal axis of the tether; and/or

in the first shape of the deformable element, the deformable element includes a first deformity extending along a first direction and a second deformity extending along a second direction different from the first direction; and/or

tensioning the tether includes tensioning a first portion of the tether coupled to a first end of the deformable element and a second portion of the tether coupled to a second end of the deformable element; and/or

a second deformable element is coupled to the tether and has a third deformed shape in the absence of applied force, and tensioning the tether to the threshold tensile force causes the second deformable element to change into a fourth shape different from the third deformed shape; and/or

the first deformed shape of the deformable element includes a first deformity extending along a first direction and the third deformed shape of the second deformable element includes a second deformity that extends along a second direction different from the first direction; and/or

during tensioning the tether, the tether is fixed to one of the prosthetic heart valve and the anchor device, and after tensioning the tether, fixing the tether to the other of the prosthetic heart valve and the anchor device; and/or

tensioning a tube received within the deformable element to the threshold tensile force to cause the tube to change from a third shape to a fourth deformed shape different from the third shape; and/or

a central portion of the tube decreases in diameter as the tube changes from the third shape to the fourth deformed shape; and/or

as the deformable element changes from the first deformed shape to the second shape, a diameter of a portion of the deformable element decreases; and/or

a clip is positioned over the central portion of the tube, and as the central portion of the tube decreases in diameter, a diameter of the clip also decreases; and/or

a radiopaque ball is received within the tether, and tensioning the tether to the threshold tensile forces causes the radiopaque ball to move from a first position relative to the tube to a second position relative to the tube; and/or

the radiopaque ball is received within an opening or detent defined along the central portion of the tube when the radiopaque ball is in the second position.

According to another aspect of the disclosure, a method of tensioning a prosthetic heart valve system, the method comprises:

positioning a tether at least partially within a heart of the patient, the tether being coupled to at least one of a prosthetic heart valve and an anchor device, and being received within a tube and defining a longitudinal axis, a radiopaque ball being positioned within the tether in a first position relative to the tube in the absence of applied forces;

tensioning the tether to a threshold tensile force to move the radiopaque ball from the first position to a second position relative to the tube, the second position being a spaced distance from the first position along the longitudinal axis; and

imaging the radiopaque ball to confirm if the radiopaque ball is at the first position or the second position; and/or

in the first position of the radiopaque ball, the radiopaque ball is received in a first opening of the tube and, in the second position of the radiopaque ball, the radiopaque ball is received in a second opening of the tube; and/or

the threshold tensile force is about 3 lb; and/or

tensioning the tether to the threshold tensile force causes a slit defined along the tube to close; and/or

tensioning the tether to the threshold tensile force cause a diameter of a first end of the tube to decrease and a diameter of the second end of the tube to increase.

According to another aspect of the disclosure, a tether system for securing a prosthetic heart valve within a patient, the tether system comprising:
a first tether portion and a second tether portion;
a deformable element secured at a first end to the first tether portion and at a second end to the second tether portion; and
the deformable element having a first diameter along a length of the deformable element in a first condition of the tether system, and a central portion of the deformable element having a second diameter less than the first diameter in a second condition of the tether system; and/or the tether system further comprises a ball received within the deformable element; and/or
the ball has a diameter larger than the second diameter of the deformable element; and/or
the deformable element has a thin section positioned between thick sections; and/or in the second condition, the thick sections have a third diameter and the thin section has a fourth diameter less than the third diameter.

According to another aspect of the disclosure, a tether system for securing a prosthetic heart valve within a patient, the tether system comprising:
a first tether portion and a second tether portion;
a deformable element secured to the first tether portion, the deformable element defining a channel and a catch protruding within the channel; and
an extension secured to the first tether portion, the extension positioned within the channel, and defining a first indent and a second indent,
wherein, the catch of the deformable element engages the first indent in a first condition of the tether system and the catch of the deformable element engages the second indent in a second condition of the tether system; and/or
the extension has a first diameter at a first end and a second diameter at a second end, the second end being greater than the first diameter; and/or
the deformable element is a sleeve; and/or
the deformable element includes a plurality of fingers.

According to another aspect of the disclosure, a tether system for securing a prosthetic heart valve within a patient, the tether system comprising:
a first tether portion and a second tether portion;
a male coupling secured to the first tether portion, a shaft extending from the male coupling; and
a female coupling secured to the second tether portion, the female coupling defining a first hole and a second hole,
wherein, the shaft is received in the first hole in a first condition of the tether system and the shaft is received in the second hole in a second condition of the tether system.

Although the subject matter herein has been described with reference to particular embodiments, it is to be understood that these embodiments are merely illustrative of the principles and applications of the subject matter described. It is therefore to be understood that numerous modifications may be made to the illustrative embodiments and that other arrangements may be devised without departing from the spirit and scope as defined by the appended claims.

1. A tether system for securing a prosthetic heart valve within a patient, the tether system comprising:
a tether defining a longitudinal axis; and
a deformable element coupled to the tether, the deformable element having a first deformed shape in a first condition of the deformable element, and having a second substantially straight shape in a second condition of the deformable element so that, when the deformable element is in the second condition, the deformable element extends in a direction along the longitudinal axis.

2. The tether system of claim 1, wherein the deformable element is configured to transition from the first condition to the second condition upon application of a threshold tensile force to the tether.

3. The tether system of claim 2, the deformable element is configured to maintain the first deformed shape when a first tensile force is applied to the tether, the first tensile force being smaller than the threshold tensile force, and the deformable element is configured to maintain the second substantially straight shape when a second tensile force is applied to the tether, the second tensile force being equal to or greater than the threshold tensile force.

4. The tether system of claim 1, wherein the deformable element is received within the tether and secured to the tether by one or more sutures.

5. The tether system of claim 1, wherein the first deformed shape includes a kink, a bend, or a curve.

6. The tether system of claim 1, wherein the deformable element is one of radiopaque or echogenic, and formed of a shape memory material.

7. The tether system of claim 1, wherein the deformable element is shape set so as to have the first deformed shape in the absence of applied force.

8. The tether system of claim 1, wherein the tether includes a first tether portion having an end coupled to a first end of the deformable element, and a second tether portion having an end coupled to a second end of the deformable element opposite the first end of the deformable element.

9. The tether system of claim 1, wherein when the deformed element has the first deformed shape, the deformed element includes a first deformity that extends along a first direction and a second deformity extending along a second direction, the first direction being transverse to the second direction.

10. The tether system of claim 1, further comprising a second deformable element coupled to the tether, the second deformable element having a first deformed shape in a first condition of the second deformable element, and having a second substantially straight shape in a second condition of the second deformable element so that, when the second deformable element is in the second condition, the second deformable element extends in the direction along the longitudinal axis.

11. The tether system of claim 10, wherein the first deformed shape of the first deformable element includes a first deformity and the second deformed shape of the second deformable element includes a second deformity, the first and second deformities extending along different directions.

12. A method of tensioning a prosthetic heart valve system, the method comprising:
positioning a tether at least partially within a heart of a patient, the tether being coupled to at least one of a prosthetic heart valve and an anchor device, a deformable element being coupled to the tether and having a first deformed shape in the absence of applied force;
tensioning the tether to a threshold tensile force and causing the deformable element, via the tensioning, to change into a second shape different than the first deformed shape; and
imaging the deformable element to confirm if the deformable element has the first deformed shape or the second shape.

13. The method of claim 12, wherein causing the deformable element to change into the second shape includes causing the deformable element to straighten so that the deformable element extends along a longitudinal axis of the tether.

14. The method of claim 13, wherein, in the first deformed shape of the deformable element, the deformable element includes a first deformity extending along a first direction and a second deformity extending along a second direction different from the first direction.

15. The method of claim 12, wherein tensioning the tether includes tensioning a first portion of the tether coupled to a first end of the deformable element and a second portion of the tether coupled to a second end of the deformable element.

16. The method of claim 12, wherein a second deformable element is coupled to the tether and has a third deformed shape in the absence of applied force, and tensioning the tether to the threshold tensile force causes the second deformable element to change into a fourth shape different from the third deformed shape.

17. The method of claim 16, wherein the first deformed shape of the deformable element includes a first deformity extending along a first direction and the third deformed shape of the second deformable element includes a second deformity that extends along a second direction different form the first direction.

18. The method of claim 12, wherein during tensioning the tether, the tether is fixed one of the prosthetic heart valve and the anchor device, and after tensioning the tether, fixing the tether to the other of the prosthetic heart valve and the anchor device.

19. The method of claim 12, further comprising:
tensioning a tube received within the deformable element to the threshold tensile force to cause the tube to change from a third shape to a fourth deformed shape different from the third shape.

20. The method of claim 19, wherein a central portion of the tube decreases in diameter as the tube changes from the third deformed shape to the fourth deformed shape.

21. The method of claim 20, wherein, as the deformable element changes from the first deformed shape to the second shape, a diameter of a portion of the deformable element decreases.

22. The method of claim 20, wherein a clip is positioned over the central portion of the tube, and as the central portion of the tube decreases in diameter, a diameter of the clip also decreases.

23. The method of claim 20, wherein a radiopaque ball is received within the tether, and tensioning the tether to the threshold tensile forces causes the radiopaque ball to move from a first position relative to the tube to a second position relative to the tube.

24. The method of claim 23, wherein the radiopaque ball is received within an opening or detent defined along the central portion of the tube when the radiopaque ball is in the second position.
